(19) United States
(12) Patent Application Publication
(10) Pub. No.: US 2024/0181001 A1
(43) Pub. Date: Jun. 6, 2024

(54) TREATMENT

(71) Applicant: Queen Mary University London, London (GB)

(72) Inventors: Karibaan Hodivala-Dilke, London (GB); Jose Manuel Felix, London (GB)

(21) Appl. No.: 18/548,793

(22) PCT Filed: Mar. 7, 2022
(86) PCT No.: PCT/GB2022/050592

§ 371 Date: Sep. 1, 2023

(30) Foreign Application Priority Data
Mar. 5, 2021 (GB) 2103122.4

(51) Int. Cl. A61K 38/12; A61K 39/00; A61P 35/00

(52) U.S. Cl. CPC A61K 38/12; A61K 39/4611; A61K 39/4631; A61K 39/4643; A61P 35/00

(57) ABSTRACT

The invention relates to the treatment of cancer in a patient with an αvβ3- and/or αvβ5-integrin targeting agent in combination with at least one immunotherapeutic agent and/or at least one chemotherapeutic agent.

### FIELD OF THE INVENTION

The invention relates to the medical use of an αvβ3- and/or αvβ5-integrin targeting agent for treatment of cancer in a patient, where the treatment further comprises administering at least one immunotherapeutic agent to the patient. The invention also relates to the medical use of an αvβ3- and/or αvβ5-integrin targeting agent for treatment of cancer in combination with at least one chemotherapeutic agent. The invention further relates to methods of treatment of cancer with the above agents and to combination products useful therein.

### BACKGROUND TO THE INVENTION

The provision of new therapies for cancer is of high importance, including cancers with poor responses to existing therapies such as immunotherapy and chemotherapy. Combination therapies able to reduce doses/side-effects of existing agents are also needed. Lung cancer is currently the leading cause of cancer mortality. Non-small cell lung cancer (NSCLC) comprises more than 80% of all lung cancers and often presents as advanced disease with poor prognosis. At diagnosis, 75% of NSCLC patients present with advanced disease (Stage III-IV) (1). These advanced cancers are inoperable.

The introduction of immunotherapy has probably contributed the most to improved treatment outcomes for NSCLC patients (2), but despite this, resistance to therapy and toxicity effects are still major obstacles. immune checkpoint blockade with anti-PD-1 (programmed cell death protein 1) and anti-CTLA-4 (Cytotoxic T-Lymphocyte Antigen 4) antibodies only provide an additional 13-month improvement in overall survival, and only 20-30% of patients respond.

More recently, combining chemotherapy, such as cisplatin, which enhances neoantigen expression, with immunotherapy has shown some promise (3), but obstacles still include: (1) lack of immunotherapy efficacy due to inadequate delivery and oxygenation (4); (2) poor T-cell infiltration into tumors (5); (3) poor intratumoral activation of T-cells (6); and (4) onset of severe autoimmune responses that can force early termination of treatment (7). Therefore, there is an urgent need to find ways to enhance the efficacy of immunotherapy for the treatment of NSCLC whilst reducing side effects.

Inadequate tumor infiltration by T cells and lower levels of PD-L1 (Programmed death ligand-1) is associated with poor response to immunotherapy (8). Increased levels of CD8+ cells or an increase in the CD8+/regulatory T cell (Tregs) ratio sensitize tumors to immunocheckpoint blockade (9). Hypoxic tumors are highly infiltrated by Tregs while CD8+ cells are absent (10). Angiogenic tumor vasculature is disorganized with irregular, leaky vessels promoting intratumoral hypoxia. Vessel co-option is a non-angiogenic mechanism by which tumor cells hijack pre-existing blood vessels to support tumor growth (11). In lungs, tumor cells can adopt vascular co-option strategies to oxygenate the tumor in addition to angiogenesis (12, 13). This observation suggests such lung cancer may well be at least partially resistant to anti-angiogenic therapies. Indeed in human primary lung cancers histopathological analysis has revealed evidence that increased levels of vessel co-optive interstitial growth patterns associate with poor outcome (14).

Previous studies have demonstrated that the combination treatment of intravenously administered low dose cilengitide (an αvβ3/αvβ5-targeting RGD-mimetic) with suboptimal doses of gemcitabine and verapamil increases blood vessel density and enhances gemcitabine metabolism, thus providing a strategy for improving cancer control whilst reducing side effects of gemcitabine (20).

There is a need for combination treatments that increase efficacy of/responsiveness to immunotherapeutics and chemotherapeutics and reduce their side effects.

### SUMMARY OF THE INVENTION

The inventors have surprisingly shown that an αvβ3- and/or αvβ5-integrin targeting agent, as illustrated by an orally available RGD-mimetic hexapeptide prodrug, 29P, is able to improve immunotherapy efficacy, and also improve efficacy of chemotherapy. Using the K-Ras^{LSL-G12D/+}; p53^{fl/fl} (KP) mouse model of NSCLC, the inventors have demonstrated that 29P sensitizes animals to standard of care cisplatin and immune checkpoint blockade using anti-PD1 and anti-CTLA, leading to a reduction in tumor burden and extended survival whilst reducing adverse side effects. 29P treatment modifies lung tumor growth patterns, leading to an increase in blood vessel density, tumor perfusion, and T-cell infiltration and activation whilst reducing hypoxia.

The effects observed are also expected to be useful to improve treatment in other cancers where immunotherapy and/or chemotherapy are employed, including cancers that are non-angiogenic and resistant to anti-angiogenic therapies. The effects observed also allow for enhancement of treatment effects of immunotherapy and/or chemotherapy such that a lower dose of immunotherapeutic agents and/or chemotherapeutic agents is able to be used therapeutically, allowing for reduction of toxicity and side-effects of such agents.

The invention thus provides an αvβ3- and/or αvβ5-integrin targeting agent for use in a method of treating a cancer in a patient, wherein said method further comprises administering at least one immunotherapeutic agent and/or chemotherapeutic agent to the patient. The invention further provides at least one immunotherapeutic agent for use in a method of treating a cancer in a patient, wherein said method further comprises administering an αvβ3- and/or αvβ5-integrin targeting agent to the patient. The invention further provides a method of treating a cancer in a patient, comprising administering an αvβ3- and/or αvβ5-integrin targeting agent and at least one immunotherapeutic agent. The invention additionally provides use of an αvβ3- and/or αvβ5-integrin targeting agent in the manufacture of a medicament for treatment of cancer, wherein the treatment further comprises administration of at least one immunotherapeutic agent. The invention also provides a combination of an αvβ3- and/or αvβ5-integrin targeting agent and at least one immunotherapeutic agent and/or at least one chemotherapeutic agent. The invention additionally provides an αvβ3- and/or αvβ5-integrin targeting agent for use in a method of treating a cancer in a patient, wherein said method further comprises administering at least one chemotherapeutic agent.

### BRIEF DESCRIPTION OF THE FIGURES

FIG. 1 illustrates that KP tumors display vessel co-option growth patterns and do not respond to VEGFR2 inhibition. (A) KP tumors show vessel co-option growth patterns: alveolar growth pattern (middle panel) and interstitial growth pattern (bottom panel). (B) Human NSCLC sections stained with hematoxylin-eosin (top panels) and double Podoplanin/CD31 immunostaining, confirming the alveolar and interstitial growth patterns occur in patients. (C) From 56 days post-viral Cre infection, KP mice were treated with DC101 (600 μg/mouse twice per week for 3 weeks). Representative images of hematoxylin-eosin and Endomucin staining of tumor-bearing lungs from KP mice treated with either placebo or DC101 (left). Quantification of number of foci, tumor burden and blood vessel density (right) confirms no significant differences. N.S. Not significant/p>0.05, unpaired Student's t-test and Mann-Whitney test, n=7 Placebo, n=8 DC101 for tumor burden and number of foci, vessel density based on n>4 fields of view per animal of n=6 animals each. Scale bars: 50 μm in IF.

FIG. 2 illustrates vascular effects of 29P treatment on KP tumors. From 56 days post-viral Cre infection, KP mice were treated with 29P (250 μg/kg twice per week for 3 weeks). (A) Representative images of hematoxylin-eosin stained lungs (left) and quantification of number of tumor foci and tumor burden (right), showing no significant differences. (B) Representative images of Podoplanin/Endomucin immunofluorescence (top) and Endomucin immunohistochemistry staining (bottom). Quantification of percentage of tumors showing the interstitial growth pattern and blood vessel density (right panel). (C) Representative images of Podoplanin/Endomucin immunofluorescence in mice treated with 29P, showing the two different vascular growth patterns. Quantification of blood vessel density (right). (D-G) Representative images (left) and quantification (right) of PE-PECAM/Endomucin double immunostaining and number of perfused vessels per field of view (D), PE-PECAM/Hoechst double immunostaining and relative Hoechst intensity (E), Glut1 immunohistochemical staining and Glut1 expression level (F), CD3 immunohistochemical staining and flow cytometry analysis (G); placebo vs 29P treated mice. N.S. Not significant/p>0.05, *p<0.05, ** p<0.01, *** p<0.001. (A,B,D-G) Unpaired Student's t-test, (C) Two-way ANOVA with Sidak's multiple comparison test, n=5-8 per treatment, vessel density based on n>4 fields of view per animal of n=7-8 animals each. Scale bars: 500 μm in IF low magnification, 50 μm in IF high magnification.

FIG. 3 illustrates that 29P treatment sensitizes KP tumors to current immunotherapies and chemotherapies. From 56 days post-viral Cre infection, KP mice were treated with Placebo (saline) or isotype antibody (100 μg/mouse), and combinations of cisplatin (6 mg/kg [Cis⁶] or 3 mg/kg [Cis³]), PD-1 mAb (200 μg/mouse), CTLA-4 mAb (100 μg/mouse), and 29P (250 μg/kg), frequency as described in the Examples. (A, B, E) Representative images of hematoxylin-eosin stained lungs and quantification of number of tumor foci and tumor burden. (C) Kaplan-Meier graph showing significant survival benefit in mice treated with the quadruple combination of Cis³, PD-1+CTLA-4 mAbs and 29P. (D) C57/BL6 mice were treated with Placebo, 29P, Cis⁶/α-PD-1+α-CTLA-4 and Cis³/α-PD-1+α-CTLA-4/29P. Quantification of mouse body weights, hematological analysis of lymphocytes and biochemical analysis of kidney damage response indicator creatinine show significantly improved parameters for the quadruple combination with low dose cisplatin (Cis³). (F) Representative images of hematoxylin-eosin stained lungs (left) and quantification of mouse body weights and tumor burden (right). N.S. Not significant/p>0.05, *p<0.05, **p<0.01, ***p<0.001. (A,B,D-F) ordinary One-way ANOVA with Tukey's multiple comparison test, for not normally distributed data Kruskal-Wallis test with Dunn's multiple comparison test, (C) Mantel-Cox test, n=5-8 per treatment. Scale bars: 5 mm in H&E staining.

FIG. 4 illustrates that 29P treatment modifies tumor growth patterns, increases blood vessel density and reduces tumor hypoxia. From 56 days post-viral Cre infection, KP mice were treated with Placebo (saline, [A]) or isotype antibody (100 μg/mouse [B]), and combinations of cisplatin (3 mg/kg [Cis3]), PD-1 mAb (200 μg/mouse), CTLA-4 mAb (100 μg/mouse), and 29P (250 g/kg) for 3 weeks and harvested after 1 further week off-treatment. Representative images of Podoplanin/Endomucin double immunofluorescence staining (A, B top row), Endomucin immunohistochemical staining (B second row), Glut1 immunohistochemical staining (B third row), or PD-11 immunohistochemical staining (B bottom row) of lungs (left) and quantification of the tumor growth patterns in mice (A, B top row), blood vessel density (B second row), Glut1 expression intensity (B third row), or number of foci showing high PD-L1 expression (B bottom row) (right). N.S. Not significant/p>0.05, *p<0.05, **p<0.01, ***p<0.001, ****p<0.0001, ordinary One-way ANOVA with Tukey's multiple comparison test, n=6-9 per treatment, vessel density based on n>5 fields of view per animal of n>6 animals each. Scale bars: 250 μm in IF.

FIG. 5 illustrates that 29P treatment induces an immunopermissive tumor microenvironment. From 56 days post-viral Cre infection, KP mice were treated with Placebo (saline, [A]) or isotype antibody (100 μg/mouse [B]), and combinations of cisplatin (3 mg/kg [Cis³]), PD-1 mAb (200 μg/mouse), CTLA-4 mAb (100 μg/mouse), and 29P (250 μg/kg) for 3 weeks and harvested after 1 further week off-treatment. (A, B) Representative images of CD3 and CD8 immunohistochemical staining of lungs (left) and quantification of the number of lesions infiltrated by CD3+ or CD8+ cells, as well as number of CD8+ cells per area (right). (C) Flow cytometry analysis of lymphoid and myeloid cell populations in tumor burdened lungs, results displayed as mean±SEM of absolute number of cells (×10⁵) per mg of tissue, NT=non tumor (tumor free lungs from control animals). N.S. Not significant/p>0.05, *p<0.05, **p<0.01, ***p<0.001. Ordinary One-way ANOVA with Tukey's multiple comparison test, for not normally distributed data Kruskal-Wallis test with Dunn's multiple comparison test, n=5-8 per treatment, for CD8+ cells/area n=5 areas per animal, n=3-7 animals per treatment.

FIG. 6 illustrates that 29P-induced effects are VEGFR2 dependent. From 56 days post-viral Cre infection, KP mice were treated with Placebo, 29P (250 μg/kg) or 29P and DC101 (600 μg/mouse) twice per week for 3 weeks. (A) Representative images of hematoxylin-eosin stained lungs (top row), Endomucin immunohistochemical staining (middle row), Podoplanin/Endomucin double immunofluorescence staining (bottom row) and quantification of tumor burden, blood vessel density, and tumor growth patterns (left). Representative images of Podoplanin/Endomucin double immunohistochemical staining [tumor lesion indicated by dashed line] and quantification of lesion size according to tumor growth pattern (right). (B, C) Representative images of Endomucin/Ki67 double (B) and Endomucin/Ki67/Podoplanin triple (C) immunofluorescence staining (upper row) and quantification of proliferating endothelial cells (below). N.S. Not significant/p>0.05, *p<0.05, **p<0.01, ***p<0.001. Unpaired Student's 1-test, n=6-8 per treatment, vessel density based on n≥5 fields of view per animal of n≥6 animals each, Ki67+ Endomucin+ based on n=10 (B)/n=2-5 (C) fields of view per animal of n=6-7 animals. Scale bars: 100 μm in IF (A, C), 50 μm in IF (B).

FIG. 7 illustrates that depletion of CD8+ cells and the IL-18-IFN-γ-CXCL9 axis reverses the anti-tumor effects of 29P. From 56 days post-viral Cre infection, KP mice were treated with isotype antibody (100 μg/mouse), and combinations of cisplatin (6 mg/kg [Cis⁶]), PD-1 mAb (200 μg/mouse), CTLA-4 mAb (100 μg/mouse), CD8α mAb (200 μg/mouse), IFN-γ mAb, anti-CXCL9 mAb, anti-IL-18 mAb and 29P (250 μg/kg) for 3 weeks, as indicated in panels. (A-E) Representative images of hematoxylin-eosin stained lungs (left) and quantification of the number of foci and tumor burden (right). (D, bottom panel) Representative images of CD8 immunohistochemistry staining (left) and quantification of the number of CD8+ cells, infiltrated into tumor lesions, per area (right). N.S. Not significant/p>0.05, *p<0.05, **p<0.01, ***p<0.001. Ordinary One-way ANOVA with Tukey's multiple comparison test, for not normally distributed data Kruskal-Wallis test with Dunn's multiple comparison test, n=7-14 per treatment.

FIG. 8, related to FIG. 1 illustrates analysis of the tumors undergoing interstitital growth patterns over time. (A) Mice bearing KP tumors were sacrificed and lungs were tharvested at 63, 70, 77 and 83 days post-viral Cre infection. Hematoxylin-eosin staining shows the increasing tumor burden (top), quantification of the number of foci and tumor burden (bottom). (B) Representative Podoplanin immunofluorescence stainings of lungs harvested at the time points shown (top) and quantification of interstitial tumor growth (bottom). *p<0.05, **p<0.01, ***p<0.001. Ordinary One-way ANOVA with Tukey's multiple comparison test, comparing all groups to 63 days, n=4-5 animals per treatment. Scale bars: 500 μm in IF.

FIG. 9, related to FIG. 2, illustrates analysis of tumor growth and tumor progression after 29P treatment. (A) Representative images of hematoxylin-eosin stained lungs of mice bearing KP tumors at 56 post-viral Cre infection. Higher magnification pictures show lung adenoma (left picture) and lung adenocarcinoma (right picture) in these KP tumors. (B) From 56 days post-viral Cre infection, KP mice were treated with 29P (250 μg/kg once per week). Representative images of hematoxylin-eosin stained lungs (top) and quantification of number of tumor foci and tumor burden (bottom), showing no significant differences. (C, D) Representative images of hematoxylin-eosin stained lungs, exhibiting different tumor grades (C) and quantification of grades after treatment with 29P, showing no difference to mice treated with placebo (D); grade 0 (hyperplasia), grades 1-2 (adenoma), grades 3-4 (adenocarcinoma) and grade 5 (desmoplastic tumors). (E) Representative images of Endomucin/α-SMA double immunofluorescence staining (left) and quantification of vessels covered by α-SMA+ perivascular cells. (F) Cell survival analysis of KP lung tumor cells after different doses of 29P. N.S. Not significant/p>0.05, *p<0.05, **p<0.01, ***p<0.001. Unpaired Student's 1-test, n=7-9 per treatment (B, E), Chi-Square test (D). Scale bars: 100 μm in low magnification, 50 μm in high magnification (C).

FIG. 10, related to FIG. 3, illustrates that 29P controls tumor growth in combination with PD-1. (A) From 56 days post-viral Cre infection, KP mice were treated with isotype antibody (100 μg/mouse), PD-1 mAb (200 μg/mouse) alone or PD-1 and 29P (250 ug/kg), frequency as described in the methods. Representative images of hematoxylin-eosin stained lungs (top) and quantification of number of tumor foci and tumor burden (bottom). (B) Cell survival analysis of human A459 cells and mouse KP lung tumor cells exposed to different concentrations of cisplatin or cisplatin and 2nM 29P. N.S. Not significant/p>0.05, *p<0.05, **p<0.01. Ordinary One-way ANOVA with Tukey's multiple comparison test, for not normally distributed data Kruskal-Wallis test with Dunn's multiple comparison test, n=9 animals per treatment, n-6-8 independent samples per condition. g.

FIG. 11, related to FIG. 5, illustrates that 29P-treatment promotes infiltration of CD3+ cells into interstitial tumor lesions. From 56 days post-viral Cre infection, KP mice were treated with isotype antibody (100 μg/mouse), and combinations of PD-1 mAb (200 μg/mouse), CTLA-4 mAb (100 μg/mouse), and 29P (250 μug/kg) for 3 weeks and harvested 1 further week after off-treatment. Representative images of CD3/Podoplanin/Endomucin triple immunofluorescence staining of lungs (top) and quantification of the number of interstitial lesions infiltrated by CD3+ cells (bottom). *p<0.05, **p<0.01. Ordinary One-way ANOVA with Tukey's multiple comparison test, n=2-23 lesions from n=6-8 animals per treatment. Scale bar: 100 μm.

### BRIEF DESCRIPTION OF THE SEQUENCES

SEQ ID NOs: 1-46 as shown in the description and sequence listing are amino acid sequences of peptides and prodrugs.

### DETAILED DESCRIPTION

It is to be understood that different applications of the disclosed products and methods may be tailored to the specific needs in the art. It is also to be understood that the terminology used herein is for the purpose of describing particular embodiments of the invention only, and is not intended to be limiting.

In addition as used in this specification and the appended claims, the singular forms “a”, “an”, and “the” include plural referents unless the content clearly dictates otherwise. Thus, for example, reference to “an αvBβ3- and/or αvβ5-integrin targeting agent” includes two or more such agents and the like.

All publications, patents and patent applications cited herein, whether supra or infra, are hereby incorporated by reference in their entirety.

### Treatment of Cancer

The invention provides a method of treating cancer in a patient comprising administering an αvβ3- and/or αvβ5-integrin targeting agent and at least one immunotherapeutic agent. The invention also provides a method of treating cancer in a patient comprising administering an αvβ3- and/or αvβ5-integrin targeting agent and at least one chemotherapeutic agent. References to treatment of cancer herein include treatment of one or more tumours. The methods of the invention may preferably comprise administering an αvβ3- and/or αvβ5-integrin targeting agent, at least one immunotherapeutic agent and at least one chemotherapeutic agent.

The invention also provides medical uses corresponding to the methods of treatment described herein and incorporating any feature described herein for the methods of the invention. The invention thus provides an αvβ3- and/or αvβ5-integrin targeting agent for use in a method of treating cancer in a patient comprising administering at least one immunotherapeutic agent and/or at least one chemotherapeutic agent. The invention also provides at least one immunotherapeutic agent and/or at least one chemotherapeutic agent for use in a method of treating cancer in a patient comprising administering at least one αvβ3- and/or αvβ5-integrin targeting agent. The invention also provides use of an αvβ3- and/or αvβ5-integrin targeting agent in the manufacture of a medicament for treatment of cancer, wherein the treatment further comprises administration of at least one immunotherapeutic agent and/or at least one chemotherapeutic agent. The invention further provides use of at least one immunotherapeutic agent and/or at least one chemotherapeutic agent in the manufacture of a medicament for treatment of cancer, wherein the treatment further comprises administration of an αvβ3- and/or αvβ5-integrin targeting agent.

αvβ3- and or avB5-Integrin Targeting Agent

The αvβ3- and/or αvβ5-integrin targeting agent may be any agent that targets αvβ3- and/or αvβ5-integrins in a manner suitable to treat cancer in combination with at least one immunotherapeutic agent and/or at least one chemotherapeutic agent, by any means. Typically, the αvβ3- and/or αvβ5-integrin targeting agent enhances the efficacy of the at least one immunotherapeutic agent and/or at least one chemotherapeutic agent in treating the cancer. The at least one immunotherapeutic agent and/or at least one chemotherapeutic agent may be unable to treat, or unable to effectively treat the cancer in the absence of the αvβ3- and/or αvβ5-integrin targeting agent.

The αvβ3- and/or αvβ5-integrin targeting agent may enhance survival of the patient from the cancer when used in combination with the at least one immunotherapeutic agent and/or at least one chemotherapeutic agent. The αvβ3- and/or αvβ5-integrin targeting agent may reduce tumor burden or reduce the number of tumour foci when used in combination with the at least one immunotherapeutic agent and/or at least one chemotherapeutic agent. The αvβ3- and/or αvβ5-integrin targeting agent may sensitise the cancer to treatment with the at least one immunotherapeutic agent and/or at least one chemotherapeutic agent. The αvβ3- and/or αvβ5-integrin targeting agent may reduce the dose of the at least one immunotherapeutic agent and/or at least one chemotherapeutic agent required for a therapeutic effect on the cancer. The αvβ3- and/or αvβ5-integrin targeting agent may thus reduce side effects and/or toxicity of the at least one immunotherapeutic agent and/or at least one chemotherapeutic agent in treatment of cancer.

The αvβ3- and/or αvβ5-integrin targeting agent may increase an anti-cancer or anti-tumour immune response when used in combination according to the invention, i.e. in combination with at least one immunotherapeutic agent and/or at least one chemotherapeutic agent. The αvβ3- and/or αvβ5-integrin targeting agent may increase infiltration of immune cells, typically activated immune cells, in the cancer or tumour. The levels of such immune cells in the cancer or tumour may thus be increased by administration the agent. The immune cells typically include T cells, preferably CD8+ T cells, typically activated CD8+ T cells. The αvβ3- and/or αvβ5-integrin targeting agent may thus increase tumour inflammation. The αvβ3- and/or αvβ5-integrin targeting agent may promote an immunopermissive tumour microenvironment. The αvβ3- and/or αvβ5-integrin targeting agent may thus promote upregulation of molecules associated with immune response in the cancer or tumour, such as molecules mediating immune cell (such as T cell) infiltration. The αvβ3- and/or αvβ5-integrin targeting agent may promote upregulation of one or more cytokines, chemokines and their receptors, such as one or more of CXCL13, CXCR5, CXCR3, CCL5, CCL9, CCL10, IFN-gamma and IL-18. Preferably, the αvβ3- and/or αvβ5-integrin targeting agent may upregulate one, two or all three of IL-18, IFN-gamma and CXCL9, which are illustrated in the Examples as having particular importance to observed effects. The αvβ3- and/or αvβ5-integrin targeting agent may upregulate molecules as described above and also increase infiltration of T cells, preferably activated CD8+ T cells.

The αvβ3- and/or αvβ5-integrin targeting agent may additionally or alternatively increase tumour perfusion. The αvβ3- and/or αvβ5-integrin targeting agent may reduce hypoxia in a cancer or tumour. The αvβ3- and/or αvβ5-integrin targeting agent may additionally or alternatively increase blood vessel density in a cancer or tumour. The αvβ3- and/or αvβ5-integrin targeting agent may additionally or alternatively promote neo-angiogenesis in a cancer or tumour, including in a non-angiogenic cancer or tumour, such as a cancer or tumour exhibiting vessel co-option.

The αvβ3- and/or αvβ5-integrin targeting agent may additionally or alternatively upregulate PD-L1 expression in the cancer or tumour.

The above functional properties of an αvβ3- and/or αvβ5-integrin targeting agent may be readily evaluated by the skilled person, including by use of the techniques and mouse models described in the Examples.

Typically, the integrin targeting agent induces altered signalling and/or gene expression but may not affect integrin adhesive function. The agent may cross-activate other receptor signalling pathways.

The targeting agent may target αvβ3- and/or αvβ5-integrin and optionally further integrins. The agent may bind one or more further integrins which are expressed or over-expressed in a cancer or tumour, such as αvβ1, α2β1, α3β1, αvβ6, αvβ8, and/or αvβ1.

Typically, the agent targets αvβ3-integrin and optionally may also target other integrins such as αvβ5-integrin. Preferably, the agent is selective for αvβ3-integrin (and optionally also for αvβ5-integrin) over other integrins and does not display high binding affinity for platelet integrins such as αIIbβ3. In some embodiments, the targeting agent may also target one or more of integrins avß6, αvβ8 and αvβ1, typically at reduced affinity as compared to αvβ3.

The agent may be any type of molecule, such as a small molecule ligand (such as an organic compound of less than 5 kDa, for example SB-273005 (CAS no. 205678-31-5), SB-267268 (CAS no. 205678-26-8) or MK-0429 (CAS no. 227963-15-7)), a peptide, a protein, an antibody, a polynucleotide, or an oligonucleotide.

Typically, the agent is a small molecule ligand, a peptide, a protein, or an antibody. Such an agent typically specifically binds directly to αvβ3- and/or αvβ5-integrin, but agents that target αvβ3- and/or αvβ5-integrin indirectly to provide for the effects described above may also be used. An agent specifically binds to a target (αvβ3- and/or αvβ5-integrin) when it binds with preferential or high affinity to that target but does not substantially bind, does not bind or binds with only low affinity to other targets. For instance, an antibody “specifically binds” a target protein when it binds with preferential or high affinity to that target protein but does not substantially bind, does not bind or binds with only low affinity to other human antigens.

An antibody binds with preferential or high affinity if it binds with a Kd of 1×10-7 M or less, more preferably 5×10-8 M or less, more preferably 1×10-8 M or less or more preferably 5×10-9 M or less. An antibody binds with low affinity if it binds with a Kd of 1×10-6 M or more, more preferably 1×10-5 M or more, more preferably 1×10-4 M or more, more preferably 1×10-3 M or more, even more preferably 1×10-2 M or more.

The antibody may be, for example, a monoclonal antibody, a polyclonal antibody, a single chain antibody, a chimeric antibody, a bispecific antibody, a CDR-grafted antibody or a humanized antibody. The antibody may be an intact immunoglobulin molecule or a fragment thereof such as a Fab, F(ab′)₂ or Fv fragment.

Preferably, the agent is a peptide. The term “peptide” as used herein is meant to encompass a chain of natural (genetically encoded), non-natural and/or chemically modified amino acid residues. The amino acid residues are represented throughout the specification and claims by either one or three-letter codes, as is commonly known in the art.

The amino acids in the sequences of peptides described herein are typically represented by a single letter as known in the art, wherein a small letter represents the corresponding amino acid in the D configuration (and a capital letter the L configuration). An asterisk symbol followed by a letter means that the corresponding amino acid is N-methylated.

A peptide described herein may be 4, 5, 6, 7, 8, 9, 10, 11, 12, 13, 14, 15, 16, 17, 18, 19, or 20 amino acids in length. Peptides described herein may be 4 to 10, 5 to 19, 5 to 18, 15 to 17, 5 to 16, 5 to 15, 5 to 14, 5 to 13, 5 to 12, 5 to 11, 5 to 10, 5 to 9, 5 to 8, or 5 to 7 amino acids in length.

Peptides described herein are typically cyclized. The terms “cyclo” or “cyclic” are used herein interchangeably and intend to indicate that the peptide is cyclized. Any type of cyclization may be applied, including but not limited to: head-to-tail cyclization, side chain to side chain, sidechain to terminal, disulfide bridge, or backbone cyclization, preferably head-to-tail cyclization. In some embodiments, the peptides comprise head-to-tail cyclization, namely having a covalent bond between the atoms of the amino terminal amino group and the carboxyl terminus of the peptide. A “c” letter followed by brackets delineating a peptide sequence means that said peptide is cyclic. Principles of design of cyclic peptides, including integrin-targeting cyclic peptides are well known in the art, and described for example in Weide et al (Topics in Curr Chemistry 2007, 272, 1-40) and Kessler (ACIE 1982, 21, 512-523).

Typical cyclic peptides targeting αvβ3- and/or αvβ5-integrin comprise the sequence motif RGD. R, and/or D may be L- or D-amino acids. Amino acids in the motif may be modified provided that this is compatible with activity of the peptide as a targeting agent e.g. the introduction of the modification should not impair the ability of the cylic peptide to target αvβ3- and/or αvβ5-integrin. For example, the length of the aliphatic chain between the alpha carbon of the amino acid to the guanidinium functional group in Arg may be altered.

The ability of a peptide to target αvβ3- and/or αvβ5-integrin can be determined using various methods. For example, the ability of peptide described herein to target αvβ3- and/or αvβ5-integrin may be determined by ELISA (see e.g. Kapp et al. Sci Rep. 2017; 7:39805), cellular assays or force field microscopy.

A cyclic peptide comprising the sequence motif RGD is typically about five to about eight amino acids in length. Such a cyclic peptide may be five, six, seven or eight amino acids in length, such as five to seven, five to six, six to seven or six to eight amino acids in length. Preferably, the peptide is a pentapeptide or a hexapeptide. The peptide may comprise two β-turns.

The above cyclic peptides may comprise at least one N-alkylated (such as N-methylated) amino acid residue, such as at least two, or at least three N-alkylations or N-methylations. The N-alkylation (such as N-methylation) may assist in constraining peptide conformation and/or increase metabolic stability. Said peptides may comprise 4 or 5 N-alkylations or N-methylations. The cyclic peptide may be a hexapeptide comprising the above specified numbers of N-alkylations or N-methylations. The N-alkylations or N-methylations may be in a position selected from the group consisting of (1,5), (1,6), (3,5), and (5,6).

The above cyclic peptides may comprise at least one D-amino acid. The cyclic peptide may be an N-methylated cyclic pentapeptide or hexapeptide comprising at least one amino acid in the D configuration. Such a peptide may comprise at least one alanine residue in the D configuration, at least one valine residue or at least one phenylalanine residue in the D configuration. The amino acid in D configuration may be at position number 1 in the peptide or in the i+1 position of a βII′ turn.

The above cyclic peptides may comprise at least one β-amino acid or γ-amino acid. The above cyclic peptides may additionally comprise at least one hydrophobic amino acid residue (such as Pro, Ile, Leu, Val, Phe, Trp, Tyr, Met), preferably selected from Val or Phe.

The peptide may be cilengitide (c(f*VRGD) or a derivative or analogue thereof, including derivatives or analogues where amino acids are changed to the L- or D-configuration, where f and/or V are substituted for alternative hydrophobic amino acids (such as Pro, Ile, Leu, Val, Phe, Trp, Tyr, Met) or for Gly, or where the peptide comprises an alternative N-alkylation to the specified N-Me or additional N-alkylations or N-methylations. Typically, a D-amino acid or Gly is provided at the i+1 position of a βII′ turn (Muller et al. Proteins: Structure, Function, and Genetics 1993, 15, 235-251).

The peptide may be one of the following peptides or a derivative or analogue thereof:

| | a. |
|---|---|
| (SEQ ID NO: 1) | |
| | *rGDA*AA; |
| | b. |
| (SEQ ID NO: 13) | |
| | *GDAA*A; |
| | c. |
| (SEQ ID NO: 2) | |
| | *aRGDA*A; |
| | d. |
| (SEQ ID NO: 3) | |
| | rG*DA*AA; |
| | e. |
| (SEQ ID NO: 4) | |
| | rGDA*A*A; |
| | f. |
| (SEQ ID NO: 5) | |
| | *vRGDA*A; |
| | g. |
| (SEQ ID NO: 6) | |
| | *fRGDA*A; |
| | h. |
| (SEQ ID NO: 7) | |
| | *rGDA*AV; |
| | i. |
| (SEQ ID NO: 8) | |
| | *GDA*AF; |
| | j. |
| (SEQ ID NO: 17) | |
| | LPPFRGDLA; |
| | k. |
| (SEQ ID NO: 18) | |
| | LPPGLRGD; |
| | l. |
| (SEQ ID NO: 19) | |
| | aAAAAA; |
| | or |
| | m. |
| (SEQ ID NO: 20) | |
| | f*VRGD. |

wherein * represents N-methylation of the following amino acid, R is arginine, G is glycine, D is aspartic acid, r is arginine in the D configuration, A is alanine, a is alanine in the D configuration, V is valine, v is valine in the D configuration, F is phenylalanine, and f is phenylalanine in the D configuration.

As discussed above the peptides of the invention are typically cyclized. Thus, the invention also relates to the cyclic peptides of SEQ ID NOS: 9 to 12, 24 to 31 and 36 to 46. By way of example, non-cyclized versions of the peptides of SEQ ID NOS: 9 to 12 are also described as SEQ ID NOS: 32-35.

The peptides of a. and c. to g. are also described in WO2019058374 and shown to exhibit improved specific properties such as high affinity, high selectivity and metabolic stability, high stability and suitability for oral administration, particularly when formulated as a prodrug. Preferred peptides were optimized to include a D-amino acid at a defined position and to include Phe or Val. A particularly preferred peptide is peptide f. (SEQ ID NO: 5).

Derivatives or analogues of the above peptides of a. to h. include peptides where an alternative D-amino acid or Gly is included at position 1 and/or wherein one or more alanine residues are substituted for other amino acids, typically for other uncharged amino acids, amino acids that do not have bulky side chains (for example Ile, Leu, Val, Met) and/or non-natural amino acids. Derivatives or analogues may include an alternative N-alkylation to the specified N-Me or additional N-alkylations or N-methylations. Where a peptide of a. to .f includes Phe or D-Phe, this may be substituted for Tyr or D-Tyr. The derivatives or analogues typically include a D-amino acid or Gly at the i+1 position of a βII′ turn (as described in Muller et al. Proteins: Structure, Function, and Genetics 1993, 15, 235-251), or at position 1 in the peptide.

Derivatives or analogues of any of the above peptides typically preserve or enhance activity of the peptide as a targeting agent, and may be validated for activity by the skilled person using methods known in the art and described in the Examples.

Also described herein are peptidomimetics of any of the above peptides. Principles for design of peptidomimetics are well known in the art. Including in the context of integrin-binding agents as described for example in Kapp et al. Sci Rep.: 2017 7:39805, Neubauer et al. J Med Chem 2014, 57, 3410 and Intervoll et al. Bioorg Med Chem Let 2006, 16, 6190-93.

Also described herein are salts of the peptides and peptidomimetics described described above, and analogs and chemical derivatives of the peptides and peptidomimetics.

As used herein the term “salts” refers to both salts of carboxyl groups and to acid addition salts of amino or guanido groups of the peptide molecule. Salts of carboxyl groups may be formed by means known in the art and include inorganic salts, for example sodium, calcium, ammonium, ferric or zinc salts, and the like, and salts with organic bases such as salts formed for example with amines such as triethanolamine, piperidine, procaine, and the like. Acid addition salts include, for example, salts with mineral acids such as, for example, acetic acid or oxalic acid. Salts describe here also ionic components added to the peptide solution to enhance hydrogel formation and/or mineralization of calcium minerals. Pharmaceutically acceptable salts include those formed with free amino groups such as those derived from hydrochloric, phosphoric, acetic, oxalic, tartaric acids, and the like, and those formed with free carboxyl groups such as those derived from sodium, potassium, ammonium, calcium, ferric hydroxides, isopropylamine, triethylamine, 2-ethylamino ethanol, histidine, procaine, and the like.

The peptides may be produced by any method known in the art, including recombinant and synthetic methods. Synthetic methods include solid phase synthesis, partial solid phase synthesis, fragment condensation, or classical solution synthesis. Solid phase peptide synthesis procedures are well known to one skilled in the art and described, for example by John Morrow Stewart and Janis Dillaha Young, Solid Phase Polypeptide Syntheses (2nd Ed., Pierce Chemical Company, 1984), Biron & Kessler (J. Org. Chem. 2005, 70, 5183-5189) and Chatterjee et al (Nature protocols, 2012, 7 (3), 432-444). In some embodiments, synthetic peptides are purified by preparative high-performance liquid chromatography (Creighton T. (1983) Proteins, structures and molecular principles. WH Freeman and Co. N.Y.).

The peptides may also be provided in the form of a prodrug, or is provided as a conjugate or fusion protein. The term “prodrug” as used herein refers to an inactive or relatively less active form of an active agent that becomes active through one or more metabolic processes in a subject. Prodrugs of peptides disclosed herein may comprise the modification of amino acids and/or amino acid residues to include an ester(s) and/or carbamate(s) of primary alcohols. In some embodiments and generally, amino side chains having amine moieties are modified into carbamates having —NHCO₂R moieties; whereas amino side chains having carboxylate moieties are modified into esters having —CO₂R moieties.

Prodrugs of the above peptides may have a net neutral charge, preferably being uncharged at physiological pH. The prodrug may comprise at least one moiety that reduces net charge of the peptide. For example, a peptide (such as an N-methylated cyclic hexapeptide) may be linked to at least one molecule that masks the charge of the amino acids in the peptide. The peptide may comprise a permeability enhancing moiety coupled to the peptide's sequence directly or through a spacer or linker. The spacer or linker may comprise a protease-specific cleavage site.

The prodrug may comprise at least one of the following moieties or groups of moieties (also described as permeability enhancing moieties):

(i) a —CO2R moiety, wherein R is alkyl;

(ii) a penta, hexyl or heptaoxycarbonyl moiety, optionally linked to an arginine residue;

(iii) a methyl ester moiety (OMe) or other alkyl ester moiety, such as a methyl or alkyl ester of Asp;

(iv) penta, hexyl or heptaoxycarbonyl and ester (OMe) moieties; and/or

(v) two penta, hexyl or heptaoxycarbonyl moieties, such as two hexyloxycarbonyl moieties.

The at least one —CO₂R moiety may be covalently linked to a nitrogen atom of at least one amino acid side chain (such as of an arginine side chain) of the peptide, preferably an N-methylated cyclic hexapeptide, such as the RGD cyclohexapeptides described by A. Schumacher-Klinger et al. (Molecular Pharmaceutics 2018, 15. 3468-3477).

The prodrug may comprise the moiety:

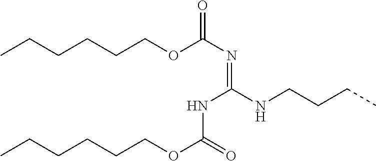

where the broken line indicates a covalent bond between the moiety and the backbone of the peptide, such as an N-methylated cyclic hexapeptide. According to some embodiments, the broken line represents a covalent bond between the moiety and an α-carbon of the peptide, such as an N-methylated cyclic hexapeptide.

R of the above moiety may be a primary alkyl group. R may be n-hexyl or any alkyl group, such as n-C₁₄H₂₉ (myristyl).

The permeability-enhancing moiety may be an oxycarbonyl moiety such as a penta, hexyl or heptaoxycarbonyl (Hoc) moiety. Hoc in all structures designates a hexyloxycarbonyl residue having the structure:

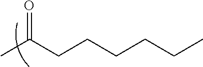

The guanidine group of Arg of the peptide may be masked with a Hoc or two Hoc moieties.

In another prodrug modification, the peptide may comprise a methyl ester moiety (OMe) or other alkyl ester moiety, such as a methyl or alkyl ester of Asp. The peptide may comprise at least one side chain having the formula CH₂COOMe.

In specific aspect, the prodrug of the peptide may have the formula:

| | c(*vR(Hoc)₂GD(OMe)A*A) |
|---|---|
| | (SEQ ID NO: 9, (also |
| | described herein as 29P)), |
| (SEQ ID NO: 10) | |
| | c(*aR(Hoc)₂GD(OMe)A*A), |
| (SEQ ID NO: 11) | |
| | c(*r(Hoc)₂GD(OMe)A*AA) |
| (SEQ ID NO: 12) | |
| | c(r(Hoc)₂GD(OMe)A*A*A), |
| (SEQ ID NO: 14) | |
| | *fR(Hoc)₂GD(OMe)A*A, |
| (SEQ ID NO: 15) | |
| | LPPFR(Hoc)₂GDLA, |
| (SEQ ID NO: 16) | |
| | LPPGLR(Hoc)₂GD, |
| (SEQ ID NO: 21) | |
| | *vR(Hoc)GD(OMe)A*A, |
| (SEQ ID NO: 22) | |
| | a*AA(Hoc)₂AAA |
| | or |
| (SEQ ID NO: 23) | |
| | f*VR(Hoc)₂GD. |

Further information on provision of preferred αvβ3- and/or αvβ5-integrin targeting peptides is provided in WO2019058374 and Weinmüller et al (Angew. Chem. Int. Ed. 2017, 56, 16405-16409), the disclosures of which (including of all designed peptides described in the documents) are incorporated by reference herein.

### Immunotherapy

The αvβ3- and/or αvβ5-integrin targeting agent is typically used in combination with at least one immunotherapeutic agent, i.e with an immunotherapy. The use of the αvβ3- and/or αvβ5-integrin targeting agent in combination with the at least one immunotherapeutic agent typically enhances the activity of the immunotherapeutic agent. The immunotherapeutic agent or immunotherapy may be any immunotherapeutic agent or immunotherapy able to treat cancer, typically by an anti-cancer or anti-tumour response. Preferably, the immunotherapeutic agent treats cancer by enhancing anti-cancer or anti-tumour response of effector T cells (typically CD8+ T cells). Effector T cell activation is normally triggered by the T cell receptor recognising antigenic peptide presented by the MHC complex. The type and level of activation achieved is then determined by the balance between signals which stimulate and signals which inhibit the effector T cell response.

The term “immune system checkpoint” is used herein to refer to any molecular interaction which alters the balance in favour of inhibition of the effector T cell response. That is, a molecular interaction which, when it occurs, negatively regulates the activation of an effector T cell. Such an interaction might be direct, such as the interaction between a ligand and a cell surface receptor which transmits an inhibitory signal into an effector T cell. Or it might be indirect, such as the blocking or inhibition of an interaction between a ligand and a cell surface receptor which would otherwise transmit an activatory signal into the effector T cell, or an interaction which promotes the upregulation of an inhibitory molecule or cell, or the depletion by an enzyme of a metabolite required by the effector T cell, or any combination thereof.

Examples of immune system checkpoints include:
a) The interaction between Indoleamine 2,3-dioxygenase (IDO1) and its substrate;
b) The interaction between PD1 and PDL1 and/or PD1 and PDL2;
c) The interaction between CTLA4 and CD86 and/or CTLA4 and CD80;
d) The interaction between B7-H3 and/or B7-H4 and their respective ligands;
e) The interaction between HVEM and BTLA;
f) The interaction between GAL9 and TIM3;
g) The interaction between MHC class I or II and LAG3; and
h) The interaction between MHC class I or II and KIR.

The immunotherapeutic agent may be any immune system checkpoint inhibitor, such as any inhibitor of any of the above checkpoints. A preferred checkpoint for the purposes of the present invention is the interaction between PD1 and either of its ligands PD-L1 and PD-L2. PD1 is expressed on effector T cells. Engagement with either ligand results in a signal which downregulates activation. The ligands are expressed by some tumours. PD-L1 in particular is expressed by many solid tumours, including melanoma, breast, bladder, colon, ovarian, uterine, and sarcoma. These tumours may therefore down regulate immune mediated anti-tumour effects through activation of the inhibitory PD1 receptors on T cells. By blocking the interaction between PD1 and one or both of its ligands, a checkpoint of the immune response may be removed, leading to augmented anti-tumour T cell responses. Therefore PD1 and its ligands are examples of components of an immune system checkpoint which may preferably be targeted by an immunotherapeutic agent in the method of the invention.

Another preferred checkpoint for the purposes of the present invention is the interaction between the T cell receptor CTLA-4 and its ligands, the B7 proteins (B7-1 and B7-2). CTLA-4 is ordinarily upregulated on the T cell surface following initial activation, and ligand binding results in a signal which inhibits further/continued activation. CTLA-4 competes for binding to the B7 proteins with the receptor CD28, which is also expressed on the T cell surface but which upregulates activation. Thus, by blocking the CTLA-4 interaction with the B7 proteins, but not the CD28 interaction with the B7 proteins, one of the normal check points of the immune response may be removed, leading to augmented anti-tumour T cell responses. Therefore CTLA4 and its ligands are examples of components of an immune system checkpoint which may preferably be targeted in the method of the invention.

The αvβ3- and/or αvβ5-integrin targeting agent may be used in combination with inhibitors of more than one immune system checkpoint, for example with an inhibitor of the interaction between PD1 and its ligands and an inhibitor of the interaction between CTLA4 and its ligands.

The immunotherapeutic agent may block or inhibit a checkpoint by binding to or otherwise modifying a component of the checkpoint, thereby blocking or inhibiting the activity of the checkpoint. The agent may be an antibody or small molecule inhibitor or other binding agent which specifically binds to a component of the checkpoint. General binding properties and examples of types of antibodies and other binding agents which specifically bind a target of interest are provided above which are also applicable to antibodies and other binding agents binding to checkpoint components. Other binding agents may include CAR T cells binding a component of the checkpoint. Examples are an antibody, CAR T cell or small molecule inhibitor of PDI binding to PD-L1 and/or PD-L2 (such as an anti-PD1 or anti-PD-L1 antibody) or an antibody, CAR T cell or small molecule inhibitor of CTLA4 binding to B7-1 and/or B7-2 (such as an anti-CTLA4 antibody). Preferred antibodies which block or inhibit the CTLA-4 interaction with B7 proteins and which may be employed in combination according to the invention include ipilumumab, tremelimumab, or any of the antibodies disclosed in WO2014/207063. Other molecules include polypeptides, or soluble mutant CD86 polypeptides. Preferred antibodies or other agents which block or inhibit the PD1 interaction with PD-L1 and which may be employed in combination according to the invention include Atezolizumab, Durvalumab, Nivolumab, Pembrolizumab, Lambrolizumab, Cemiplimab, Pidilzumab, Toripalimab, and AMP-224. Anti-PD-L1 antibodies include MEDI-4736 and MPDL3280A.

The above immunotherapeutic agents are described for combination with any αvβ3- and/or αvβ5-integrin targeting agent described herein, preferably a cyclic peptide of about five to about eight amino acids in length comprising the sequence RGD as described above, such as cilengetide or any of the peptides and prodrugs of SEQ ID NOs: 1-23 and derivatives and analogs thereof, and peptidomimetics thereof, particularly preferably the peptide of SEQ ID NO: 5 or the prodrug of SEQ ID NO:9 or a derivative, analog, or peptidomimetics of either thereof. The above immunotherapeutic agents are further described for combination with an above-described αvβ3- and/or avB5-integrin targeting agent and any chemotherapeutic agent described below, particularly preferably cisplatin, carboplatin, pemetrexed or a derivative of any thereof

### Chemotherapy

The αvβ3- and/or αvβ5-integrin targeting agent may additionally or alternatively be used in combination with at least one chemotherapeutic agent. Preferably, the αvβ3- and/or αvβ5-integrin targeting agent may be used in combination with at least one chemotherapeutic agent and at least one immunotherapeutic agent. The use of the αvβ3- and/or αvβ5-integrin targeting agent in combination with the at least one chemotherapeutic agent typically enhances the activity of the chemotherapeutic agent.

The αvβ3- and/or αvβ5-integrin targeting agent may be used in combination with any chemotherapeutic agent, such as any chemotherapeutic agent useful in treatment of a cancer of interest. The skilled person is able to select a chemotherapeutic agent suitable for treatment of a particular type of cancer. Chemotherapeutic agents, such as anticancer agents, include: alkylating agents including including platinum coordination complexes such as cisplatin (cis-DDP) and carboplatin, nitrogen mustards such as mechlorethamine (HN2), cyclophosphamide, ifosfamide, melphalan (L-sarcolysin) and chlorambucil; ethylenimines and methylmelamines such as hexamethylmelamine, thiotepa; alkyl sulphonates such as busulfan; nitrosoureas such as carmustine (BCNU), lomustine (CCNU), semustine (methyl-CCNU) and streptozocin (streptozotocin); and triazenes such as decarbazine (DTIC; dimethyltriazenoimidazole-carboxamide); Antimetabolites including gemcitabine and folic acid analogues such as methotrexate (amethopterin), pemetrexed; pyrimidine analogues such as fluorouracil (5-fluorouracil; 5-FU), floxuridine (fluorodeoxyuridine; FUdR) and cytarabine (cytosine arabinoside); and purine analogues and related inhibitors such as mercaptopurine (6-mercaptopurine; 6-MP), thioguanine (6-thioguanine; TG) and pentostatin (2′-deoxycoformycin); Natural Products including vinca alkaloids such as vinblastine (VLB) and vincristine; epipodophyllotoxins such as etoposide and teniposide; antibiotics such as dactinomycin (actinomycin D), daunorubicin (daunomycin; rubidomycin), doxorubicin, bleomycin, plicamycin (mithramycin) and mitomycin (mitomycin C); enzymes such as L-asparaginase; and biological response modifiers such as interferon alphenomes; miscellaneous agents including anthracenedione such as mitoxantrone and anthracycline; substituted urea such as hydroxyurea; methyl hydrazine derivative such as procarbazine (N-methylhydrazine, MIH); and adrenocortical suppressant such as mitotane (o,p′-DDD) and aminoglutethimide; taxol and analogues/derivatives; and hormone agonists/antagonists such as flutamide and tamoxifen.

Preferred chemotherapeutic agents include alkylating agents, in particular cisplatin, carboplatin and derivatives thereof, and antimetabolites, such as gemcitabine and pemetrexed, and a combination of any thereof, such as cisplatin, carboplatin or a derivative thereof and pemetrexed or a derivative thereof.

In some embodiments, an αvβ3- and/or αvβ5-integrin targeting agent is used in combination with at least one chemotherapeutic agent without the additional use of an immunotherapeutic agent. In such embodiments, the at least one chemotherapeutic agent may be an alkylating agent, in particular cisplatin, carboplatin and derivatives thereof, or may be pemetrexed or a derivative thereof, or a combination of any thereof, and the method does not comprise use of gemcitabine or does not comprise use of any other chemotherapeutic agent. In such embodiments, the αvβ3- and/or αvβ5-integrin targeting agent may further be a peptide of a. to m. above or a cyclized version thereof, preferably a peptide of f. or a cyclized version thereof. In such embodiments, additionally or alternatively the cancer may be a non-angiogenic cancer, such as a cancer exhibiting vessel co-option, as discussed below. The cancer may be refractory to the chemotherapeutic agent in the absence of the αvβ3- and/or αvβ5-integrin targeting agent. The cancer may be an NSCLC as discussed below.

### Cancer

The cancer may be any cancer or tumour. The cancer or tumour may be any cancer or tumour previously described for treatment with immunotherapy and/or chemotherapy.

The cancer or tumour is typically a solid cancer or tumour. The solid cancer or tumour may for example be breast, pancreatic (such as pancreatic ductal), bladder, renal, liver, colon, lung, prostate, head and neck, ovarian, uterine, squamous cell skin, mesothelioma, sarcoma or melanoma. The cancer may be a haematological cancer, such as Hodgkin's lymphoma.

The cancer or tumour may be poorly responsive, non-responsive or refractory to treatment with an immunotherapeutic agent and/or chemotherapeutic agent described above. The cancer or tumour may be poorly responsive, non-responsive or refractory to treatment with a checkpoint inhibitor described above, such as one targeting the interaction of PD-1, CTLA-4 or PD-L1 with a ligand. The cancer or tumour may be previously described as unsuitable for treatment with an immunotherapeutic agent and/or chemotherapeutic agent described above based on side-effects and/or toxicity at a therapeutic dose. The cancer or tumour may additionally or alternatively be poorly responsive, non-responsive or refractory to treatment with an anti-angiogenic agent.

The cancer or tumour may be non-angiogenic. Thus, the cancer or tumour may have inadequate vascularisation. The cancer or tumour may not display neo-vascularisation. The cancer or tumour may display hypoxia. The cancer or tumour may have aberrant perfusion. The cancer or tumour may exhibit vessel co-option.

The cancer or tumour is preferably lung cancer, most preferably NSCLC. The NSCLC may be advanced NSCLC, such as Stage III or IV NSCLC. The cancer may be small cell lung cancer. The lung cancer may be unresectable. The lung cancer may be non-angiogenic and/or may exhibit vessel co-option. The lung cancer may comprise alveolar and/or interstitial tumour growth. The lung cancer may be a primary lung cancer or any secondary cancer that has spread to the lung such as a breast or pancreatic cancer.

The patient is typically human. However, the patient may be another mammalian animal, such as a commercially farmed animal, such as a horse, a cow, a sheep, a fish, a chicken or a pig, a laboratory animal, such as a mouse or a rat, or a pet, such as a guinea pig, a hamster, a rabbit, a cat or a dog.

The patient may be previously diagnosed with cancer or exhibit one or more symptoms of cancer. The patient may have been previously treated with an immunotherapy and/or chemotherapy described herein, and may have been determined to be poorly responsive or non-responsive to said treatment. The patient may have been determined as having cancer that is non-angiogenic and/or exhibits vessel co-option.

### Pharmaceutical Composition

The agents described herein may be formulated in pharmaceutical compositions. The above methods and medical uses typically comprise administration of a pharmaceutical composition. The compositions may comprise, in addition to the therapeutically active ingredient(s), a pharmaceutically acceptable excipient, carrier, diluent, buffer, stabiliser or other materials well known to those skilled in the art. Such materials should be non-toxic and should not interfere with the efficacy of the active ingredient. The pharmaceutical carrier or diluent may be, for example, an isotonic solution.

The term “pharmaceutically acceptable” means approved by a regulatory agency such as a Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic compound is administered. Such pharmaceutical carriers can be sterile liquids, such as water and oils, including those of petroleum, animal, vegetable or synthetic origin, such as peanut oil, soybean oil, mineral oil, sesame oil and the like, polyethylene glycols, glycerine, propylene glycol or other synthetic solvents. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene glycol, water, ethanol and the like. The composition, if desired, can also contain minor amounts of wetting or emulsifying agents, or pH buffering agents such as acetates, citrates or phosphates. Antioxidants such as ascorbic acid or sodium bisulfite; chelating agents such as ethylenediaminetetraacetic acid; and agents for the adjustment of tonicity such as sodium chloride or dextrose are also envisioned.

The precise nature of the carrier or other material may depend on the route of administration, e.g. oral, intravenous, cutaneous or subcutaneous, nasal, intramuscular and intraperitoneal routes. Examples of suitable pharmaceutical carriers are described in “Remington's Pharmaceutical Sciences” by E. W. Martin. Such compositions will contain a therapeutically effective amount of the peptides according to the invention, preferably in a substantially purified form, together with a suitable amount of carrier so as to provide the form for proper administration to the subject. Examples of suitable compositions and methods of administration are provided in Esseku and Adeyeye (2011) and Van den Mooter G. (2006).

For example, solid oral forms may contain, together with the active substance, diluents, e.g. lactose, dextrose, saccharose, cellulose, corn starch or potato starch; lubricants, e.g. silica, talc, stearic acid, magnesium or calcium stearate, and/or polyethylene glycols; binding agents; e.g. starches, gum arabic, gelatin, methylcellulose, carboxymethylcellulose or polyvinyl pyrrolidone; disaggregating agents, e.g. starch, alginic acid, alginates or sodium starch glycolate; effervescing mixtures; dyestuffs; sweeteners; wetting agents, such as lecithin, polysorbates, laurylsulphates; and, in general, non-toxic and pharmacologically inactive substances used in pharmaceutical formulations. Such pharmaceutical preparations may be manufactured in known manner, for example, by means of mixing, granulating, tabletting, sugar-coating, or film-coating processes.

Oral formulations include such normally employed excipients as, for example, pharmaceutical grades of mannitol, lactose, starch, magnesium stearate, sodium saccharine, cellulose, magnesium carbonate, and the like. These compositions take the form of solutions, suspensions, tablets, pills, capsules, sustained release formulations or powders and contain 10% to 95% of active ingredient, preferably 25% to 70%. Where the pharmaceutical composition is lyophilised, the lyophilised material may be reconstituted prior to administration, e.g. a suspension. Reconstitution is preferably effected in buffer.

Capsules, tablets and pills for oral administration to an individual may be provided with an enteric coating comprising, for example, Eudragit “S”, Eudragit “L”, cellulose acetate, cellulose acetate phthalate or hydroxypropylmethyl cellulose.

Liquid dispersions for oral administration may be syrups, emulsions or suspensions. The syrups may contain as carriers, for example, saccharose or saccharose with glycerine and/or mannitol and/or sorbitol.

Suspensions and emulsions may contain as carrier, for example a natural gum, agar, sodium alginate, pectin, methylcellulose, carboxymethylcellulose, or polyvinyl alcohol. The suspensions or solutions for intramuscular injections may contain, together with the active substance, a pharmaceutically acceptable carrier, e.g. sterile water, olive oil, ethyl oleate, glycols, e.g. propylene glycol, and if desired, a suitable amount of lidocaine hydrochloride.

Solutions for intravenous administration or infusion may contain as carrier, for example, sterile water or preferably they may be in the form of sterile, aqueous, isotonic saline solutions.

For suppositories, traditional binders and carriers may include, for example, polyalkylene glycols or triglycerides; such suppositories may be formed from mixtures containing the active ingredient in the range of 0.5% to 10%, preferably 1% to 2%.

The pharmaceutical composition (such as a composition for oral administration) may comprise absorption enhancers. The pharmaceutical composition may comprise lipids. The pharmaceutical composition may comprise self nano-emulsifying drug delivery systems (SNEDDS) or Pro-NanoLiposphere (PNL). The term SNEDDS (self nano-emulsifying drug delivery systems) as used herein refers to anhydrous homogeneous liquid mixtures, composed of oil, surfactant, drug, and/or cosolvents, which spontaneously form transparent nanoemulsion. The term PNL (Pro-NanoLiposphere) as used herein refers to a delivery system based on a solution containing the drug, triglyceride, phospholipid, surfactants, and a water miscible organic solvent.

### Administration and Dose

An agent described herein or pharmaceutical composition comprising said agent is administered to treat cancer. Administration is typically in a “therapeutically effective amount”, this being sufficient to show benefit to the individual, e.g. an effective amount to ameliorate one or more symptoms, to enhance survival, to induce or prolong remission, or to delay relapse or recurrence.

The agent or pharmaceutical composition may be administered by any route, including an oral, intravenous, cutaneous or subcutaneous, nasal, intramuscular or intraperitoneal route.

The dose of each agent may be determined according to various parameters, especially according to the substance used; the age, sex, weight and condition of the individual to be treated; the route of administration; and the required regimen. A physician will be able to determine the required route of administration and dosage for any particular individual. The amount of the agent which will be effective in the treatment of a particular disorder or condition will depend on the nature of the disorder or condition, and can be determined by standard clinical techniques. In addition, in vitro assays may optionally be employed to help identify optimal dosage ranges. The precise dose to be employed in the formulation will also depend on the seriousness of the disease or disorder, and should be decided according to the judgment of the practitioner and each patient's circumstances. Toxicity and therapeutic efficacy of the agents described herein can be determined by standard pharmaceutical procedures in cell cultures or experimental animals, e.g., by determining the IC50 (the concentration which provides 50% inhibition) and the LD50 (lethal dose causing death in 50% of the tested animals) for a subject compound. The data obtained from these cell culture assays and animal studies can be used in formulating a range of dosage for use in humans.

A typical daily dose of an agent described herein is from about 0.1 to 50 mg per kg of body weight dependent on the nature of the agent and the conditions mentioned above. A general dose amount described in the art for administration of pharmaceutical compositions comprising peptides ranges from about 0.1 μg/kg to about 20 mg/kg body weight. The amount of the active ingredient may be in the range of from about 10 to 5000 μg/kg. The dose may be provided as a single dose or may be provided as multiple doses, for example taken at regular intervals. The dosage can be administered, for example, in weekly, biweekly, monthly or bimonthly regimens. Examples of the techniques and protocols mentioned above can be found in Remington's Pharmaceutical Sciences, 20th Edition, 2000, pub. Lippincott, Williams & Wilkins.

The αvβ3- and/or αvβ5-integrin targeting agent, at least one immunotherapeutic agent and/or the at least one chemotherapeutic agent may be administered simultaneously, separately, or sequentially. Each agent may be administered by the same or a different administration route. Each agent may be administered at different stages of a treatment comprising administration of the agents.

The administration of the αvβ3- and/or αvβ5-integrin targeting agent may permit reduction of the dose of the at least one immunotherapeutic agent and/or the at least one chemotherapeutic agent required for therapeutic efficacy, and thus beneficially reduce side effects and/or toxicity of such agents at higher doses. The invention thus also provides a method of reducing the dose of at least one immunotherapeutic agent and/or at least one chemotherapeutic agent required for treatment of a cancer, comprising administering an αvβ3- and/or αvβ5-integrin targeting agent in combination with said at least one immunotherapeutic agent and/or at least one chemotherapeutic agent. The method may comprise administering a first dose of the at least one immunotherapeutic agent and/or at least one chemotherapeutic agent in a combination as described above and then administering a second, lower dose of the at least one immunotherapeutic agent and/or at least one chemotherapeutic agent in a combination as described above and determining that the second, lower dose has therapeutic efficacy, optionally further determining that the second, lower dose has reduced side effects and/or toxicity.

In the methods of the invention, the αvβ3- and/or αvβ5-integrin targeting agent is administered at a dose effective to induce one or more of the functional properties of such agents as described above, and to enhance efficacy of the at least one immunotherapeutic agent and/or at least one chemotherapeutic agent. The dose is typically effective to alter αvβ3- and/or avß5 integrin signaling without inhibiting integrin adhesion.

Where the αvβ3- and/or αvβ5-integrin targeting agent is a cyclic peptide of about five to about eight amino acids in length comprising the sequence RGD as described above, such as cilengetide or any of the peptides and prodrugs of SEQ ID NOs: 1-23 and derivatives and analogs thereof, and peptidomimetics thereof, the peptide may be administered at a dose ranging from about 0.1 mg/kg to about 10 mg/kg of the subject weight. A peptide may be administered at a dose ranging from about 0.01, 0.05, 0.1, 0.5, 0.7, 1, or 2 mg/kg of the subject weight, to about 0.05, 0.1, 0.5, 0.7, 1, 2, 5, 10, 15, 20, 50, 100, 250, or 500 mg/kg of the subject weight. A peptide may be administered at a dose ranging from 0.1, 1, 10, 20, 30, 50, 100, 200, 400, 500, 700, 900 or 1000 ng/kg of the subject weight, to about 100, 200, 400, 500, 700, 900, 1000, 1200, 1400, 1700, or 2000 ng/kg of the subject weight. A peptide may be administered at a dose ranging from about 20 ng/kg to about 100 ng/kg of the subject weight. A peptide may be administered in a human dose amount corresponding to a mouse dose amount of about 50 ug/kg to about 400 μg/kg, such as about 50 μg/kg to about 300 μg/kg, about 100 μg/kg to about 300 μg/kg, or about 100 μg/kg to about 250 μg/kg. Human dose amounts below 50 μg/kg or above 400 μg/kg, such as a dose amount of about 10 μg/kg to about 700 μg/kg, may also be administered,

A peptide may be orally administered at a dose ranging from about 0.001 mg/kg to about 500 mg/kg of the subject weight, for example from about 0.1 mg/kg to about 500 mg/kg of the subject's body weight.

### Combination

The αvβ3- and/or αvβ5-integrin targeting agent is administered in the methods and medical uses of the invention combination with at least one immunotherapeutic agent and/or at least one chemotherapeutic agent. In a related aspect, the invention provides a combination of an αvβ3- and/or αvβ5-integrin targeting agent and at least one immunotherapeutic agent and/or at least one chemotherapeutic agent, as a product per se. The above combination may take the form of a composition comprising the αvβ3- and/or αvβ5-integrin targeting agent and the at least one immunotherapeutic agent and/or at least one chemotherapeutic agent, or a product comprising different dose forms of the integrin targeting agent and the at least one immunotherapeutic agent and/or at least one chemotherapeutic agent. The combination may be a kit comprising the avB3- and/or avB5-integrin targeting agent and the at least one immunotherapeutic agent and/or at least one chemotherapeutic agent, optionally with instructions for administration in a method of the invention. The at least one immunotherapeutic agent and/or at least one chemotherapeutic agent may be for administration simultaneously, separately or sequentially with the αvβ3- and/or αvβ5-integrin targeting agent.

### EXAMPLES

The following Examples are provided to illustrate the invention.

### Materials and Methods

### Conditional Mouse Model of Non-Small Cell Lung Cancer

Kras^{LSL-G12D/+}; Trp53^{flox/flox} (referred to as KP) were used to generate lung adenocarcinomas by administering Ad-Cre (Cre recombinase Adenovirus) 5×10⁷ PFU as previously described (DuPage et al, 2009; Cortez-Retamozo et al, 2013; Pfirsche et al, 2016). Mice were treated after 56 days post-adeno viral infection using different combinations: Cisplatin 6 mg/Kg (Cis⁶) or Cisplatin 3 mg/Kg (Cis³) in combination or not with 29P once a week; α-PD-1 200 μg/mouse and α-CTLA-4 100 ug/mouse twice a week or the combinations of cisplatin/29P with immunotherapies; anti-VEGFR2 (DC101) 600 μg/mouse twice a week or the combination DC101/29P twice a week.

### Xenograft Model

C57/BL6J were injected subcutaneously with 0.5×10⁶ Lewis Lung Carcinoma (LLC) cells into the flank to generate LLC-subcutaneous tumors, which display tumor angiogenesis. Tumor growth was measured by using calipers three times per week, and tumor volume was calculated using the following formula: Tumor volume=Length×width×width×0.52. When the tumor reached 50 mm³, mice bearing LLC tumors were treated with either isotype or DC101 800 μg/mouse twice per week.

### Treatments

Cisplatin (100 mg/100 ml, Teva), Verapamil (40 mg/5 ml Zolvera, Rosemant) were purchased from Barts Hospital's pharmacy and used within 3 months. 29: c(*vRGDA*A), and its prodrug 29P, c(*vR(Hoc)₂GD(OMe)A*A) were purchased from Horst Kessler Lab. PD-1 mAb (clone—29F.1A12) (Bio X Cell) 200 μg/mouse was administered twice a week over 3 weeks.

CTLA-4 mAb (clone—9D9) (Bio X Cell) 100 μg/mouse was administered twice a week over 3 weeks. Rat IgG2a isotype control, anti-trinitrophenol (Clone 2A3) (Bio X Cell) 100 μg/mouse was administered twice a week during 3 weeks. Cisplatin alone treatment was administered once per week. 29P was administered once a week if in combination with cisplatin, or twice a week if in combination with immunotherapy, neutralizing antibodies or DC101. For multicombination treatment 29P and cisplatin were given on the same day once a week, and immunotherapy twice a week 2 days and 6 days after 29P/Cisplatin administration.

### Cell Lines

The lung adenocarcinoma cell line KP53 F1 was derived from lung tumors of C57BL/6 KP mice and was kindly provided by Dr. S A. Quezada, UCL Cancer Centre (University College London, UK).

A549 and LLC cells were purchased from ATCC. Mouse Lung Endothelial cells (EC) were obtained and cultured as previously described (34).

### Cell Survival Assays:

A549 or KP cell survival was analyzed using the CellTiter96® Aqueous One Solution Reagent (Promega #G3582) according to the manufacturer's instructions. Briefly, 1000 cells were seeded into 96-well plated. Cells were treated the following day and the solution reagent was added after 72 hours of treatment. Plates were read using a wavelength of 490 nm.

### VEGFR2 Inactivation

For VEGFR2 inactivation, KP mice were administered with anti-VEGFR2 (clone DC101, Bio X Cell) twice per week during three weeks.

### In Vivo CD8+ Cell Depletion

In order to deplete CD8+ cells, KP mice were administered with anti-CD8α mAb intraperitoneally (clone YTS 169.4, BioXcell) twice per week after the first day of treatment and until the end of the experiments.

### In Vivo Interferon-γ, CXCL9 and IL-18 Inactivation

In order to deplete IFN-γ, KP mice were administered with anti-IFN-γ mAb intraperitoneally (XMG1.2, Bio X Cell) twice per week after the first day of treatment and until the end of the experiments. To deplete CXCL9, KP mice were administered with anti-CXCL9 mAb intraperitoneally (MIG-2F5.5, Bio X Cell) twice per week after the first day of treatment and until the end of the experiments. To deplete IL-18, KP mice were administered with anti-IL-18 mAb intraperitoneally (Clone YIGIF73-1G7, Bio X Cell) twice per week after the first day of treatment and until the end of the experiments.

### Immunohistochemistry and Immunofluorescence

Tissues were fixed in formalin for 24 hours and transferred to 70% ethanol. Tissues were paraffin embedded, sectioned, dewaxed, and unmasked in boiling 10 mM citrate buffer pH 6.0. Sections were washed three times in PBS, blocked in 5% normal goat serum for 1 hour, and incubated with primary antibody: rat monoclonal endomucin (Santa Cruz-sc-65495), rabbit polyclonal anti-Glut1(Abcam-ab652), rabbit polyclonal carbonic anhydrase IX (Novus Biologicals-NB100-417), mouse monoclonal anti-alpha-smooth muscle actin Cy3-conjugated (Sigma-Aldrich-C6198), rabbit monoclonal Ki67 (Abcam-ab16667), rabbit polyclonal anti-CD3 (DAKO, A045201-2), rat monoclonal anti CD3, (BioRad IBL-3/16), rabbit polyclonal anti-CD8 (abcam, ab203035), rat monoclonal anti-CD8a (ThermoFisher, 4SM15), rat monoclonal anti-F4/80 (abcam, ab6640), rat monoclonal anti-Ly6G (abcam, ab25377). Sections were then washed and incubated for 45 minutes with secondary fluorophore or biotinylated secondary antibody. For immunohistochemistry of tissue sections, enzymatic Avidin-Biotin Complex (ABC)-3,3′diaminobenzidine (DAB) staining (Vector Laboratories) was used. Nuclei were counterstained with hematoxylin.

### Vascular Perfusion

Vascular perfusion was visualized by injecting mice via the tail vein with PE-conjugated 20 μg of mouse monoclonal anti-PE-PECAM (Biolegend-102408) 10 min prior to culling. Tumors were snap frozen, sectioned and stained for endomucin. The total number of perfussed vessels (PE-PECAM positive) and the % of double-positive blood vessels provided an indication of blood vessel perfusion.

### Vessel Permeability

Extravascular diffusion was tested by injecting mice via the tail vein with Hoechst dye (0.4 mg, Sigma-Aldrich) 1 min prior to culling and anti-PE-PECAM as above. Tumors were snap frozen. 100 μm thick sections were analyzed using Axioplan microscopy (LSM510ME-TA, Carl Zeiss). Mean pixel number for PE-PECAM and Hoechst staining for each 100 μm stack was calculated using ImageJ software.

### Phenotypic Analysis of Lungs Bearing KP Tumors by Flow Cytometry

Tumors were minced and incubated at 37° C. for 20 min in an enzymatic cocktail containing DNase (0.5 mg/ml, Sigma). Liberase (2 mg/ml, Sigma) in HBSS (Sigma) was used to make a single cell suspension for pancreatic tumors. For breast tumors, collagenase/dispase (1 mg/ml, Sigma) in PBS, was used to make a single cell suspension.

Cells were passed through a 70 μM filter (BD Biosciences), washed in PBS supplemented with 2% foetal bovine serum and 2 mM EDTA, counted and used immediately for flow cytometry. Before cells were stained with specific antibodies, nonspecific binding sites were blocked with Fcγ R III/II TruStain fcX (93, Biolegend). Staining was performed in PBS supplemented with 2% foetal bovine serum and 2 mM EDTA.

The following fluorochrome-conjugated antibodies were used: anti-CD45 (30-F11), anti-CD3 (145-2C11), anti-CD4 (L3T4), anti-CD8 (53-6.7), anti-CD69 (H1.2F3), anti-CD44 (IM7), anti-CD62L (MEL-14), anti-CD19 (6D5), anti-CD11b (M1/70), anti-F4/80 (BM8) from Biolegend; anti-PD1 (RMP1-30), anti-Gr1 (RB6-8C5) and anti-Ly6C (HK1.4) from eBioscience. Fixable viability dye (FVD) (eBioscience) was used to discriminate between live and dead cells. Acquisition and analyses were performed on a BD LSRII system using BD FACSDIVA software (BD Biosciences). The percentage of cells were analysed using FlowJo software (version 10.0.8 tree Star). Dead cells were excluded from the analysis on the basis of FVD and SSC gating. Cell doublets were excluded from the analysis by gating for FSC area versus FSC width. CD45+ cells were used to include leukocytes only in the data analysis.

### RNA Extraction and RNAseq Analysis

RNA was extracted from frozen lungs bearing KP tumors treated with either placebo or 29P; KP cells treated with either placebo or 29 and MLECs treated or not with 29. In the 3 cases, the extraction of RNA was performed according the manufacturer's instructions (Qiagen, Manchester, UK). Lung bearing KP tumors were digested before extraction using a polytron homogenizer. RNA-Seq was performed by Barts and the London Genome Centre on the Illumina NextSeq 500 platform.

### Ethical Regulations

All procedures were approved by our local animal ethics committee, Queen Mary University of London, and were executed in accordance with United Kingdom Home Office regulations.

Statistical Analysis Unless otherwise indicated, data are shown with individual animals plotted as single data points, bars indicating mean, and error bars indicating standard error of the mean. All data were analysed using GraphPad Prism software. Data were first assessed for normality of distribution. For comparison of 2 groups with normally distributed data, unpaired two-tailed Student's 1-test was used; for data where distribution was not normal, the Mann-Whitney test was used. For comparison of >2 groups, one-way or two-way ANOVA were used for normally distributed data, the Kruskall-Wallis for not normally distributed data. All means were compared against each other, unless otherwise indicated in the figure legend. P values were adjusted with post hoc tests for multiple comparisons. The Chi-square test was used to compare observed histological tumor grades and the Mantel-Cox to assess differences in survival. A difference in mean was considered statistically significant if p<0.05. Individual tests used and n numbers are indicated in each figure legend.

### Example 1—Lung Cancers in Kras^{LSL-G12D/+}; p53^{fl/fl} Mice Display Vessel Co-Option Growth Patterns and Do Not Respond to Anti-VEGFR2 Therapy

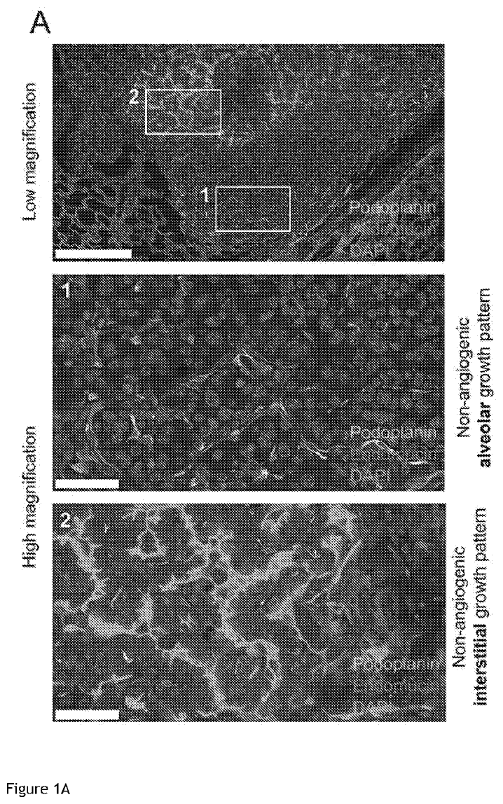

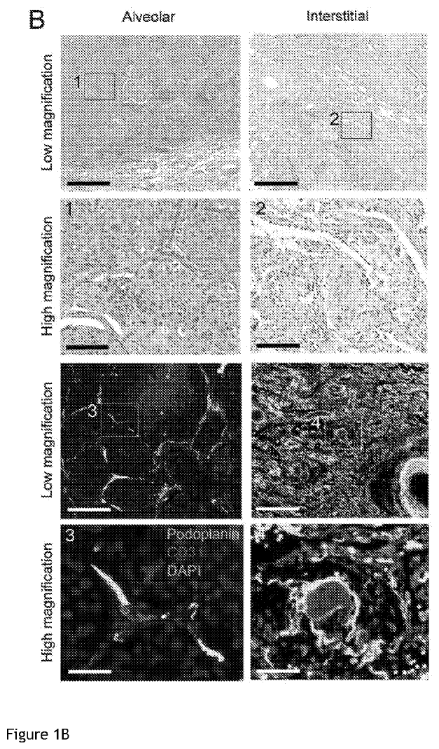

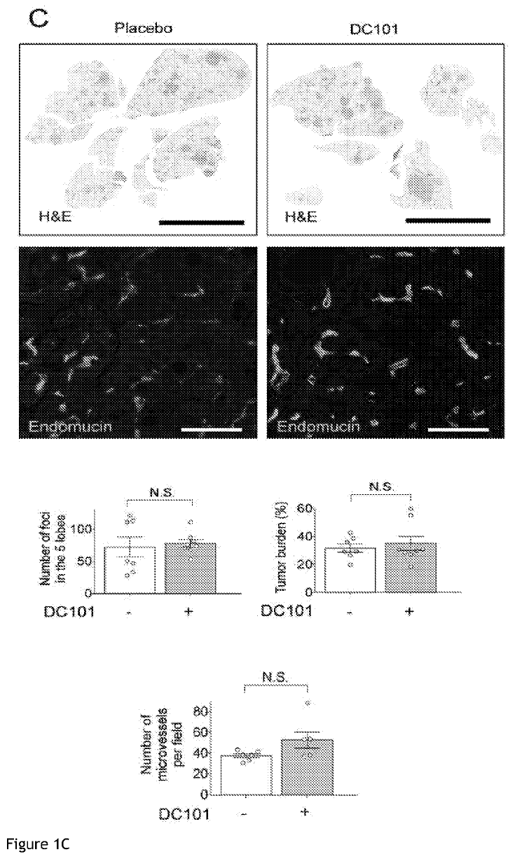

To explore the effect of vascular modulation with 29P treatment on lung cancer we first defined the vascular growth patterns in tumor bearing Kras^{LSL-G12D/+}; p53^{fl/fl} (KP) mice. Double immunofluorescence analysis of Podoplanin and Endomucin protein expression, staining lung parenchyma and endothelium respectively, demonstrated that KP mice display non-angiogenic alveolar and interstitial tumor growth patterns. Alveolar growth patterns involve tumor cell growth within the alveoli, resulting in compressed interstitial space and blood vessels, whilst interstitial growth patterns are characterized by tumor cell growth within the interstitial space rather than in the alveoli (FIG. 1A). In further results, the presence of tumors with interstitial growth pattern increased between 63 and 84 days post adeno-viral Cre administration, indicating a correlation of interstitial growth pattern and tumor progression. Double immunostaining for Podoplanin and the endothelial cell marker CD31 revealed similar patterns of interstitial growth in human non-small cell lung cancer samples (FIG. 1B), highlighting the suitability of the KP mouse model for NSCLC research. In line with the observed bias towards tumor vessel co-option rather than angiogenic vessel growth, we found that treatment of tumor burdened KP mice with anti-VEGFR2 (DC101 (Rockwell, P., Mol. Cell. Differ., 3: 91-109, 1995)) antibody had no significant effect on the number of lung tumor foci, overall tumor burden or number of microvessels per field (FIG. 1C). Together, these data suggested that vascularization of KP lung tumors was similar to that of human non-small cell lung cancer and that these were not responsive to anti-angiogenic treatment using anti-VEGFR2 antibodies.

### Example 2—29P Treatment Modifies NSCLC Growth Patterns and Increases Blood Vessel Density

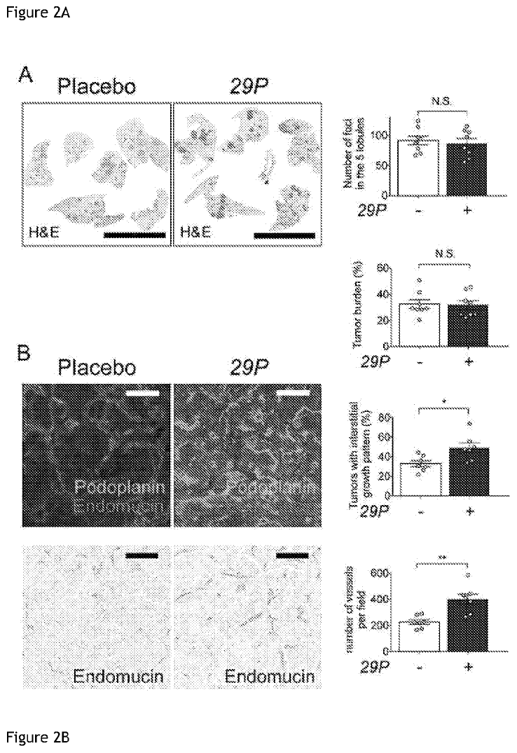

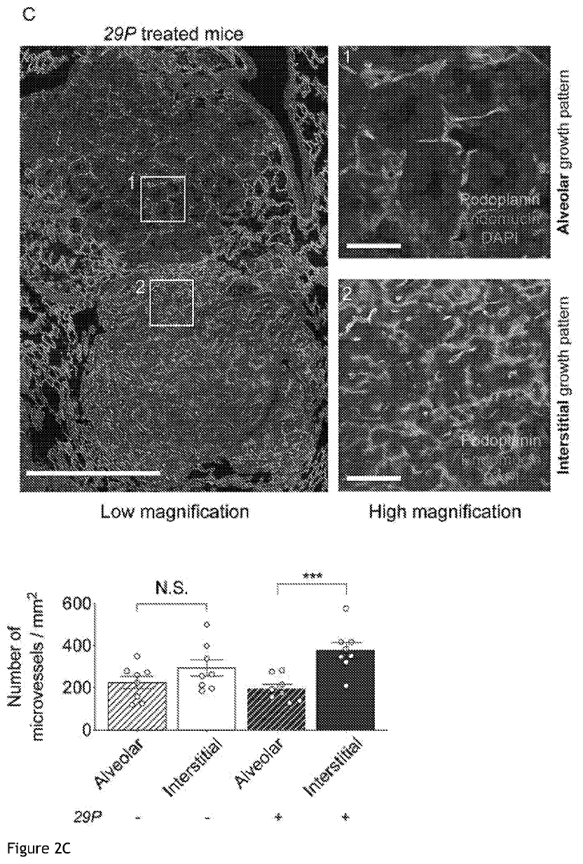

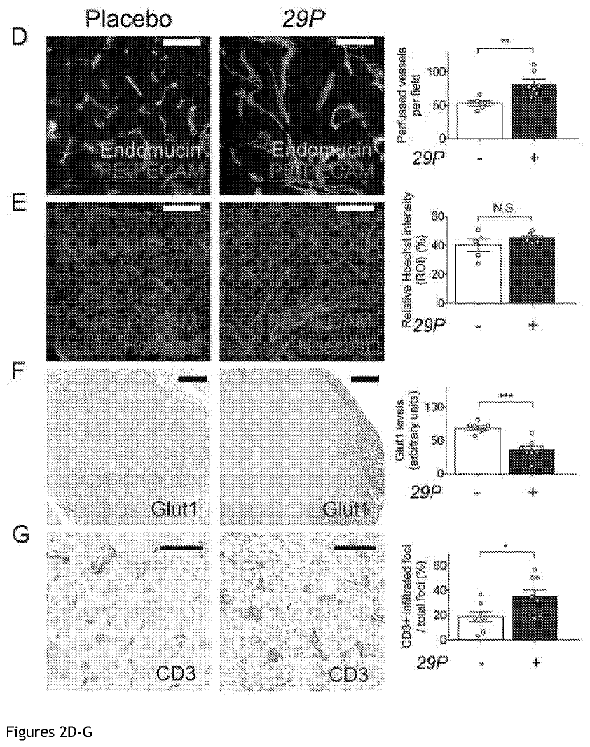

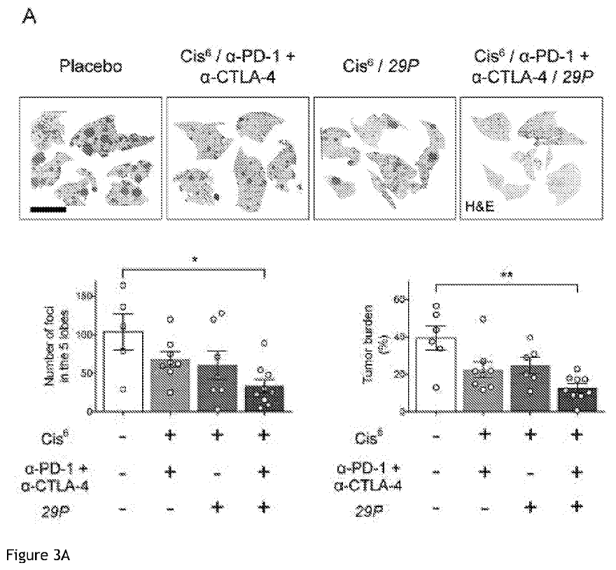

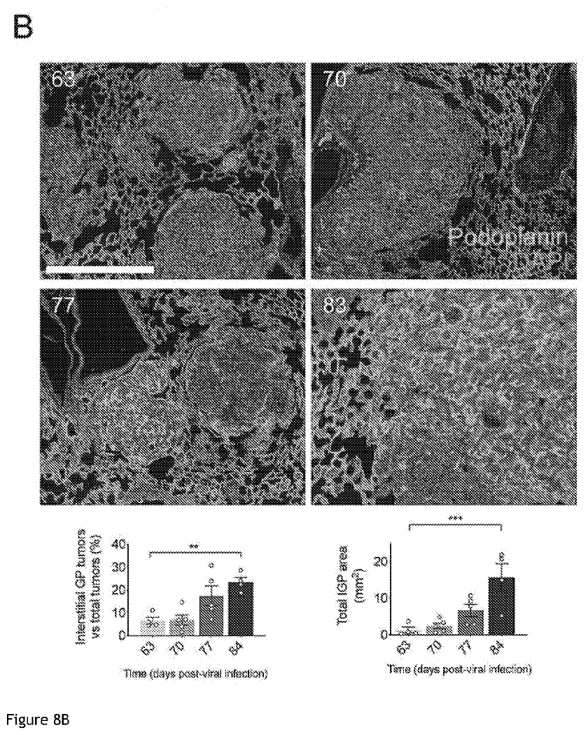

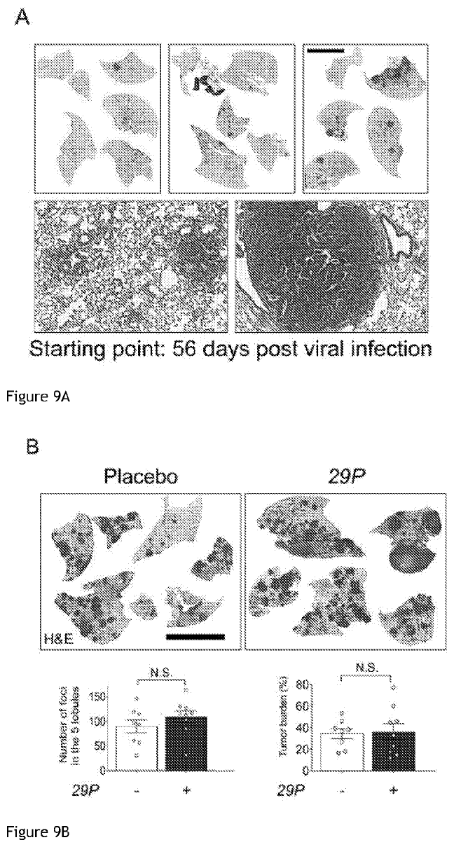

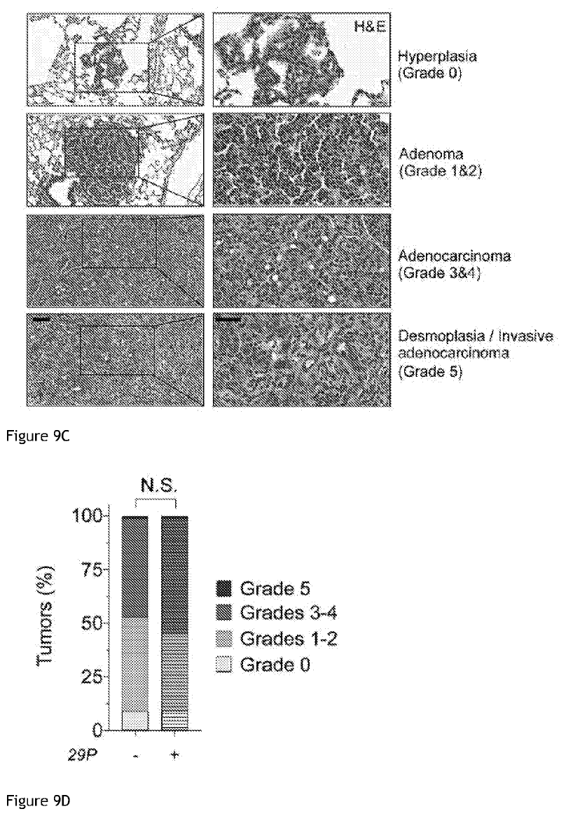

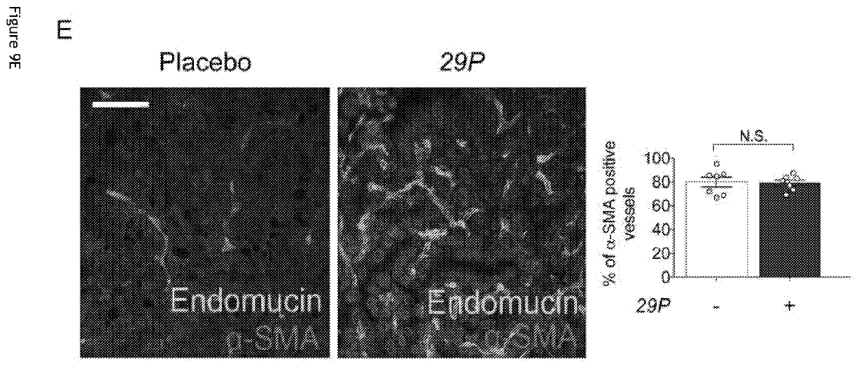

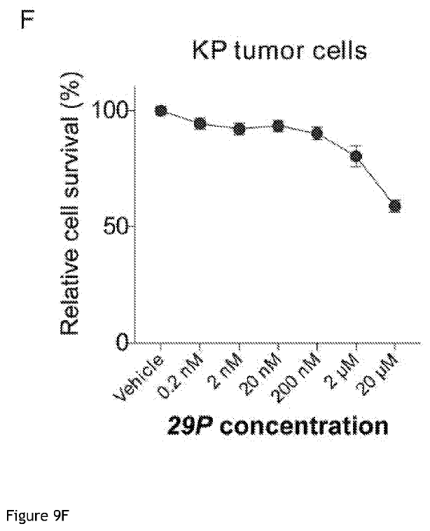

To examine the effect of 29P treatment on NSCLC growth, tumor-burdened KP mice were treated with 29P (250 μg/kg) and tumor burden and tumor blood vessels assessed at the experimental end point, 4 weeks after treatment commenced. The starting point for the treatments and the end point were chosen according previous publications with this experimental model (22-24). Histological analysis showed that 29P treatment did not affect the number of foci or tumor burden (FIG. 2A, FIG. 9B), but increased both the percentage of foci with interstitial tumor growth patterns and the total number of tumor blood vessels per field (FIG. 2B). Further immunofluorescence analysis revealed that blood vessel density was not altered by 29P treatment in foci with alveolar growth patterns, but was increased significantly in foci with interstitial tumor growth patterns (FIG. 2C). No effect was observed on either tumor progression (FIG. 9C, D) or percentage of αSMA-positive blood vessels, an indicator of blood vessel maturation (FIG. 9E). Additionally, treatment with 29P in vitro had no significant effect on KP tumor cell survival at low doses (0.2 nM-200 nM), equivalent to the dose given in vivo (Supplementary FIG. 9F). At a functional level, 29P treatment also resulted in: (1) an increase in the percentage of perfused vessels (FIG. 2D), (2) no apparent effect on vessel permeability as detected by leakage of Hoechst dye (FIG. 2E), (3) decreased levels of tumor hypoxia evaluated by Glut1 staining (FIG. 2F), and (4) an increase in the number of CD3+ cells assessed histologically and also and=increase in the percentage of CD45+ cell infiltration that were CD3+ in KP tumors by flow cytometry (FIG. 2G).

Taken together, these data demonstrated that 29P treatment modifies tumor vascularization by specifically increasing the number of tumor lesions that display the interstitial growth pattern which shows vascular co-option rather than angiogenic growth. Treatment with 29P also increased blood vessel density in these tumors and this corresponded with an increase in vessel perfusion, reduction in hypoxia and increase in CD3+ cell infiltration.

### Example 3—29P Treatment Enhances Immune Checkpoint Blockade and Cisplatin Treatment of NSCLC in KP Mice

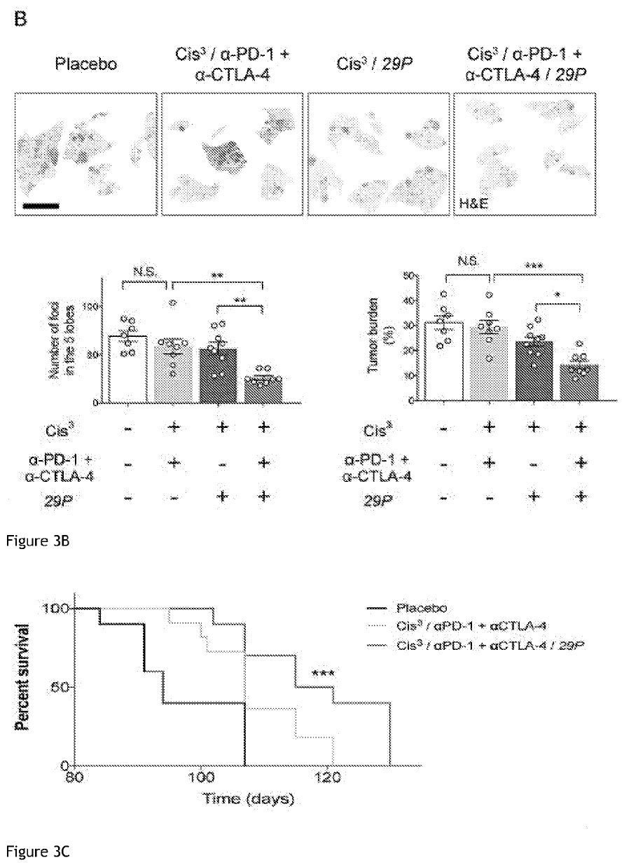

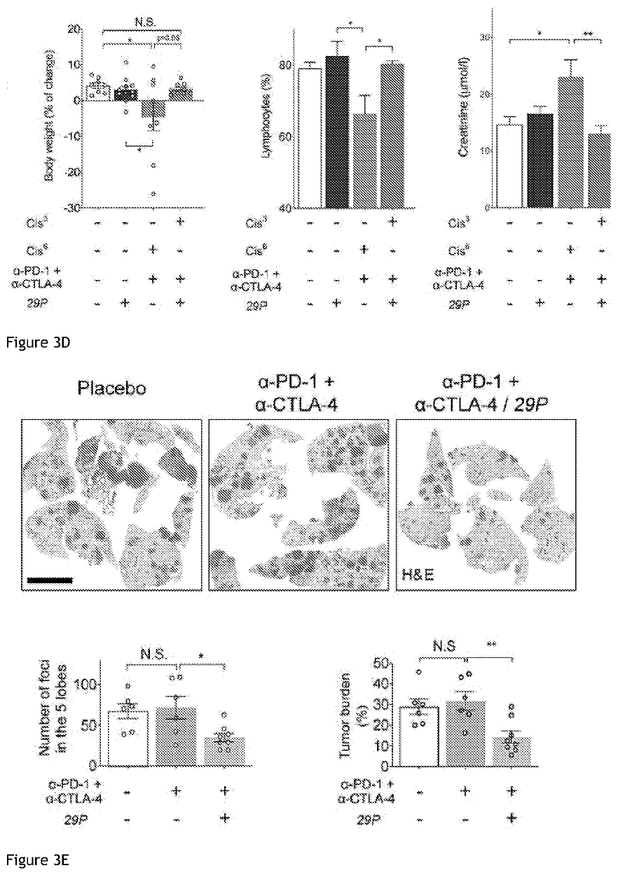

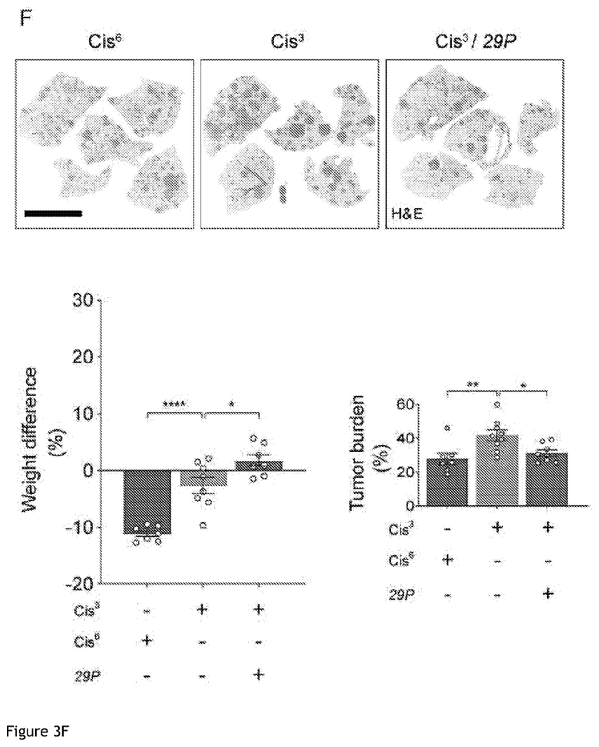

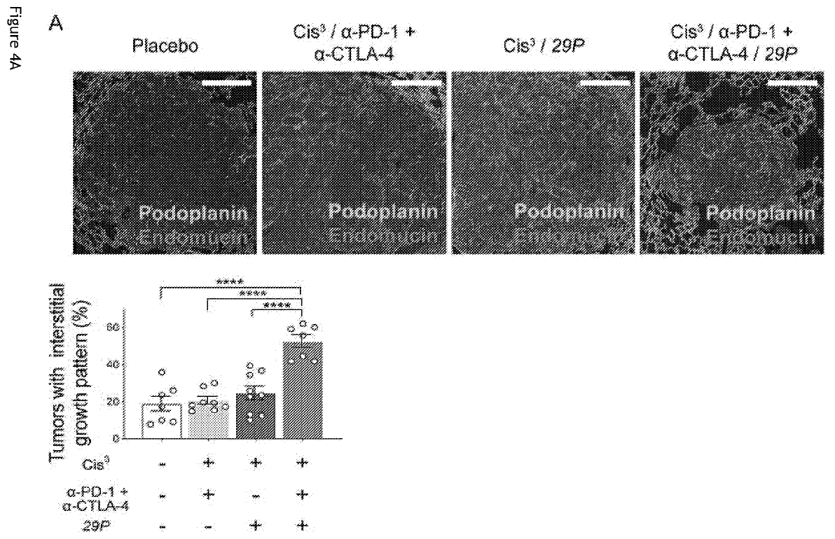

Platinum based therapies such as cisplatin are part of the first line chemotherapy and are also used in combination with immunotherapies for NSCLC treatment (25). Because hypoxia and absence of anti-tumor immune infiltrate are key obstacles of effective immunotherapy and 29P treatment improved these, we hypothesized that combining 29P treatment with chemo/immunotherapy agents would increase their efficacy. Indeed we found that KP mice treated with a multicombination of immunotherapies (anti-PD-1 and anti-CTLA-4 antibodies), Cis⁶ (cisplatin 6 mg/kg) and 29P had a significantly reduced number of foci and significantly lower tumor burden in comparison with KP mice treated with either Cis⁶/29P or Cis⁶/anti-PD-1/anti-CTLA-4 antibodies (FIG. 3A). Importantly, the same multicombination treatment, but using a lower dose of cisplatin (3 mg/kg, Cis³), also reduced tumor burden and numbers of foci whilst increasing the survival of the mice significantly compared with mice treated with Cis³/29P or Cis^{3/}anti-PD-1/anti-CTLA-4 (FIGS. 3 B and C). Lowering cisplatin doses to from 6 to 3 mg/kg resulted in overcoming the issues of toxic side effects of cisplatin. For example, while combination treatment with Cis⁶/anti-PD-1/anti-CTLA-4 reduced animal body weight, induced lymphopenia and caused high creatinine levels, indicative of kidney dysfunction, treatment with Cis³/anti-PD-1/anti-CTLA-4 and 29P restored body weight, lymphocyte numbers and creatinine levels to that of placebo or single agent 29P treated mice (FIG. 3D).

To explore whether 29P sensitized tumors to cisplatin and/or anti-PD-1/anti-CTLA-4 antibody treatment, we combined 29P with either cisplatin or single immunotherapies separately. Although treatment with anti-PD-1 plus anti-CTLA-4 antibodies did not reduce tumor growth, as described previously (23), administration of 29P in combination with anti-PD-1 and anti-CTLA-4 antibodies reduced the number of tumour foci and tumor burden (FIG. 3E). 29P treatment was also sufficient to sensitize tumors to anti-PD-1 monotherapy albeit a more modest effect than 29P with anti-PD-1 plus anti-CTLA-4 (FIG. 10A). To demonstrate better the utility of 29P, we compared the anti-tumor efficacy and adverse side effects in KP mice treated with either Cis⁶, Cis³ and Cis³/29P. Results indicate that Cis⁶ treatment has a clear anti-tumor effect, but the mice suffer a greater level of weight loss side effect. However, Cis³/29P improves the anti-tumor effect to similar levels as those obtained with Cis6 but without apparent adverse weight loss side effects (FIG. 3F). This raised the question of whether 29P could directly affect tumor cell survival in combination with cisplatin. To address this, we performed in vitro experiments and treated either human lung adenocarcinoma (A549) or mouse lung adenocarcinoma (KP) cells with increasing concentrations of cisplatin alone or in combination with 29P. Using the MTS cell viability assay, we found cisplatin cytotoxicity to be independent of 29P addition, suggesting that 29P does not directly alter the anti-tumoral effects of cisplatin (FIG. 10B). Together, these data established that in vivo treatment with 29P improves the efficacy of cisplatin in combination with immunotherapy, whilst reducing side effects, and provides a possible opportunity to increase the therapeutic effects of immunotherapy in NSCLC.

### Example 4—Treatment with 29P Switches Immunosuppressive Tumors into Immunopermissive Tumors

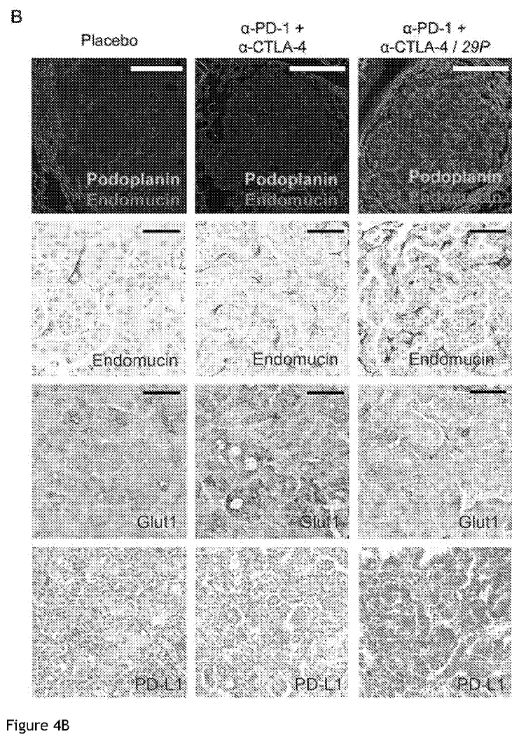

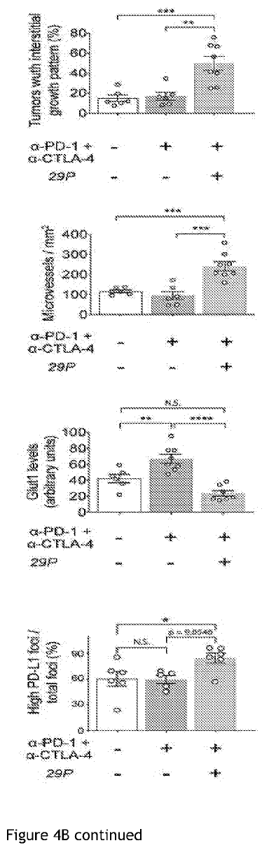

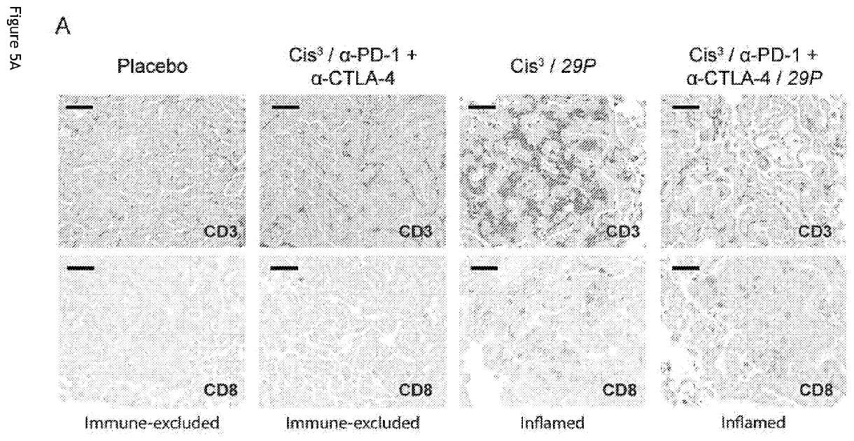

We next wanted to further explore the mechanisms underlying the enhanced therapeutic effect seen in the combination treatments. Similar to the effect of 29P alone, tumors in KP mice treated with the quadruple combination of Cis³/anti-PD-1/anti-CTLA-4 antibodies and 29P or the triple combination of anti-PD-1/anti-CTLA-4 immunotherapy and 29P showed increased percentages of tumors with an interstitial growth pattern (FIG. 4A). This treatment also enhanced blood vessel density and reduced tumor hypoxia, in comparison with mice treated with either placebo or anti-PD-1/anti-CTLA-4 antibodies alone (FIG. 4B).

On the other hand, we observed increased levels of PD-L1 protein in KP tumors treated with the combination of anti-PD-L1/anti-CTLA-4 and 29P in comparison with anti-PD-1/anti-CTLA-4-treated mice or isotype treated mice (FIG. 4C).

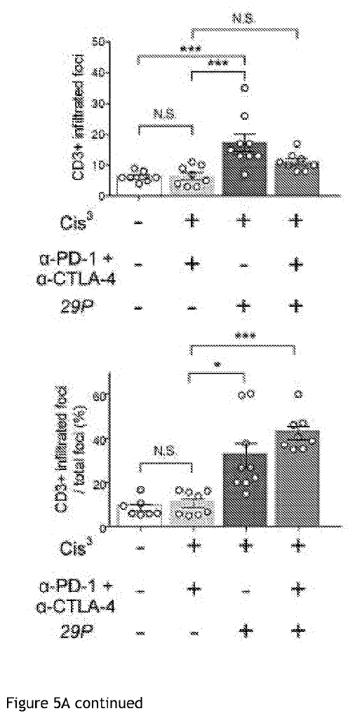

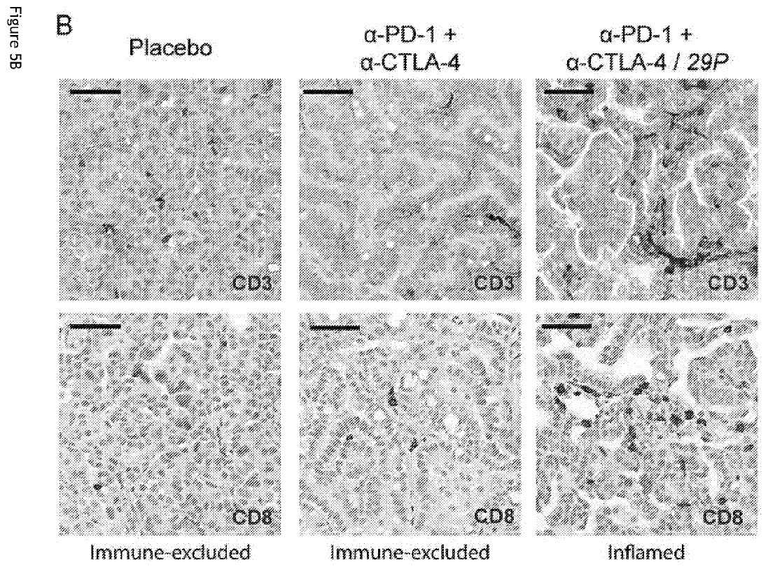

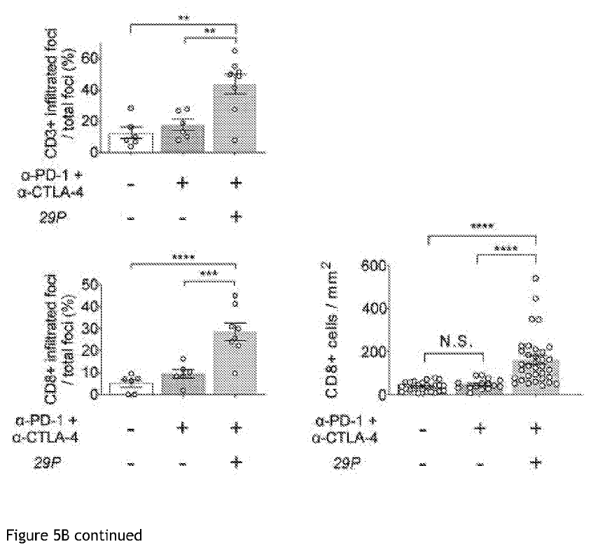

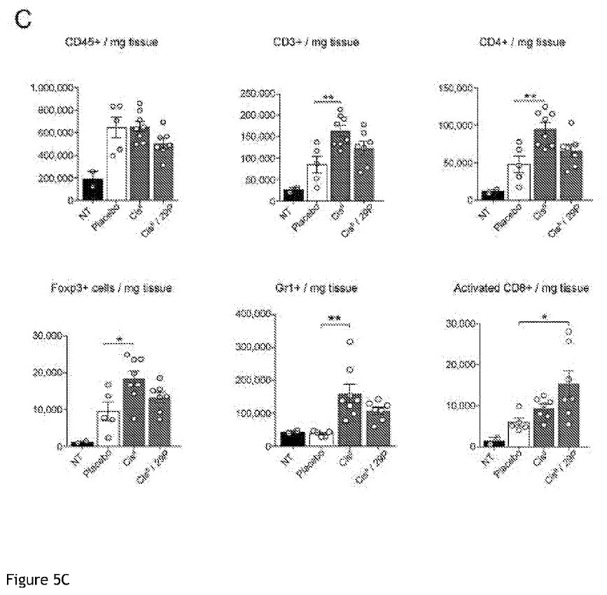

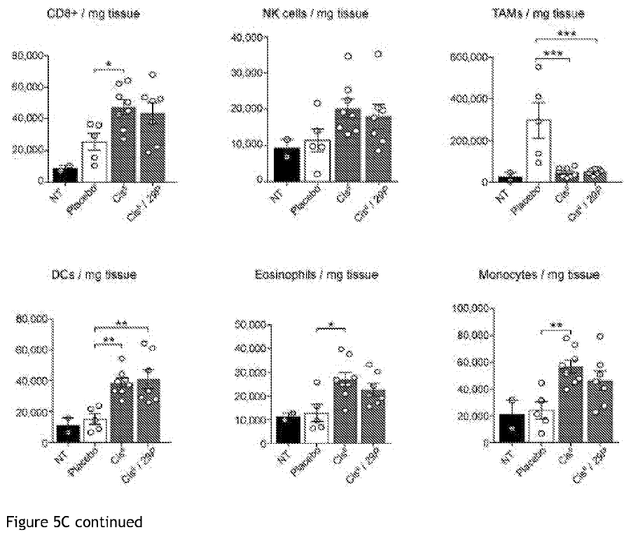

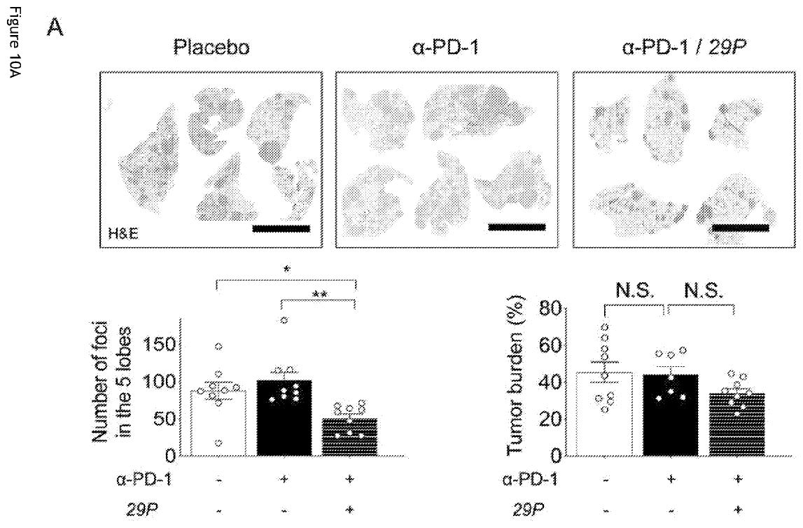

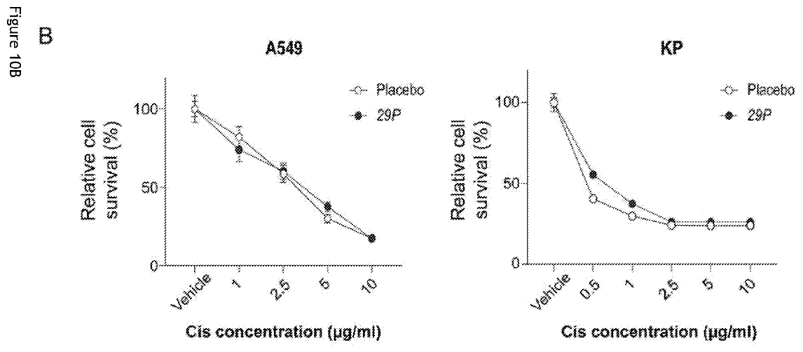

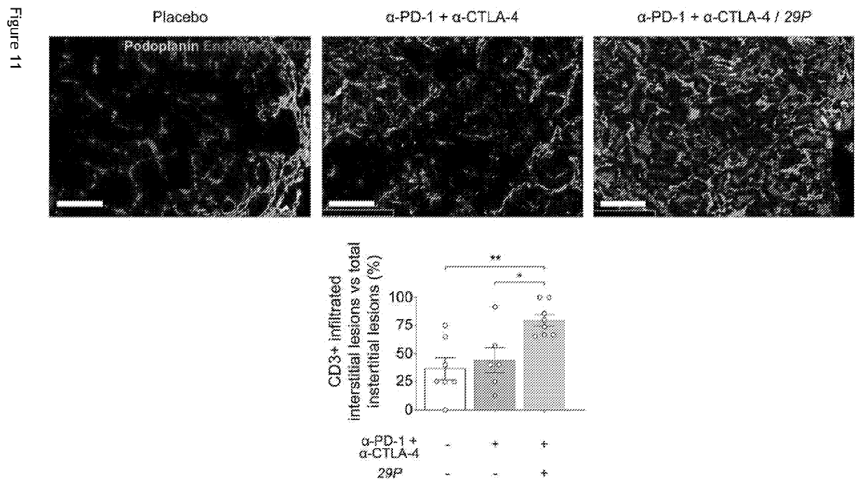

Strategies that enhance the number of infiltrating T-cells in tumors, switching immune-excluded, ‘cold’, tumors into inflamed, ‘hot’, tumors, is a major goal in current cancer research as it is widely believed that this is required for immunotherapy to fulfil its potential (2). Excitingly, we found that the enhanced interstitial growth patterns, blood vessel density and effects on reduced hypoxia in KP mice treated with 29P correlated with an increase in CD3+ and CD8+ T-cell infiltration either in combination with Cis³ alone or in combination with Cis³/anti-PD-1/anti-CTLA-4 antibodies (FIG. 5A). In the absence of Cis³, combination treatment of KP mice with 29P/anti-PD-1/anti-CTLA-4 antibodies also enhanced CD3+ and CD8+ T-cell tumor infiltration, switching immune-excluded tumors into inflamed tumors (FIG. 5B). Performing triple-immunofluorescence staining for Podoplanin, Endomucin and CD3, we show that CD3+ cell infiltration is significantly enhanced in foci with interstitial growth pattern (FIG. 11A), suggesting that the enhanced vascularization induced by 29P is associated with switching ‘cold’ tumors to ‘hot’ tumors.

Examination of whole KP tumor burdened lung cell suspensions by flow cytometry identified that cisplatin treatment increased T cell (CD4+ and CD8+) and also myeloid cell (Gr1+, Natural killer cells, dendritic cells, monocytes) numbers, despite a reduction in tumor associated macrophages. Combination treatment with low-dose cisplatin (Cis³) and 29P reduced CD3+ cell numbers, especially the regulatory T cell (T_{reg}) population, whereas no reduction in CD8+ cell numbers were observed, indicating a low ratio of T_{regs}: CD8+ cells. Moreover, numbers of activated CD8+ cells (CD8+CD44+CD62−) were increased after combined Cis³/29P treatment (FIG. 5C). Gr1+ neutrophils and monocytes were decreased while dendritic cells and natural killer cell numbers were unaffected in mice after Cis³/29P treatment in comparison with Cis⁶ alone (FIG. 5C).

Overall, treatment of KP mice with 29P depleted the inhibitory immune cells, induced by cisplatin treatment, resulting in relatively higher activation of CD8+ cells, generating an immunopermissive, ‘hot’ tumor microenvironment.

### Example 6—Enhanced Immunotherapy Efficacy After 29P Treatment is Dependent on VEGFR2-Mediated Angiogenesis

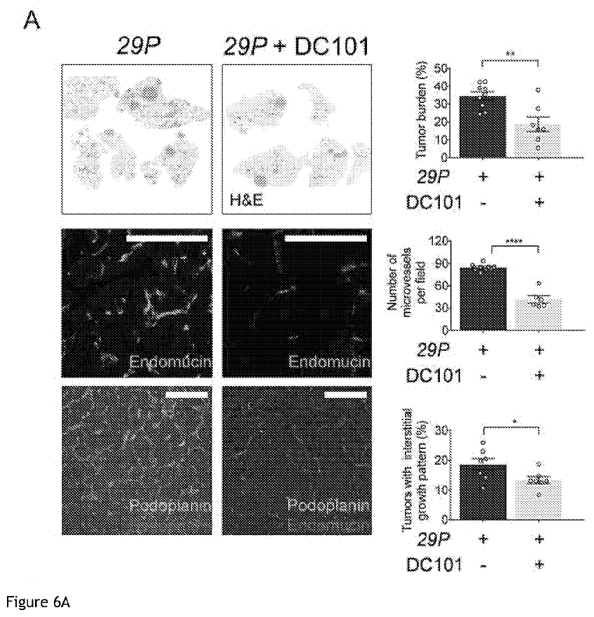

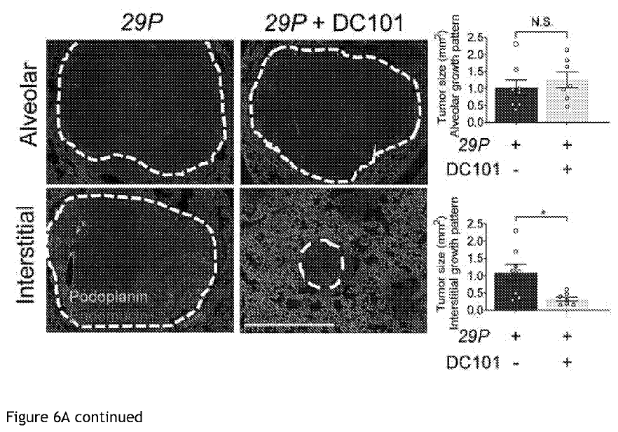

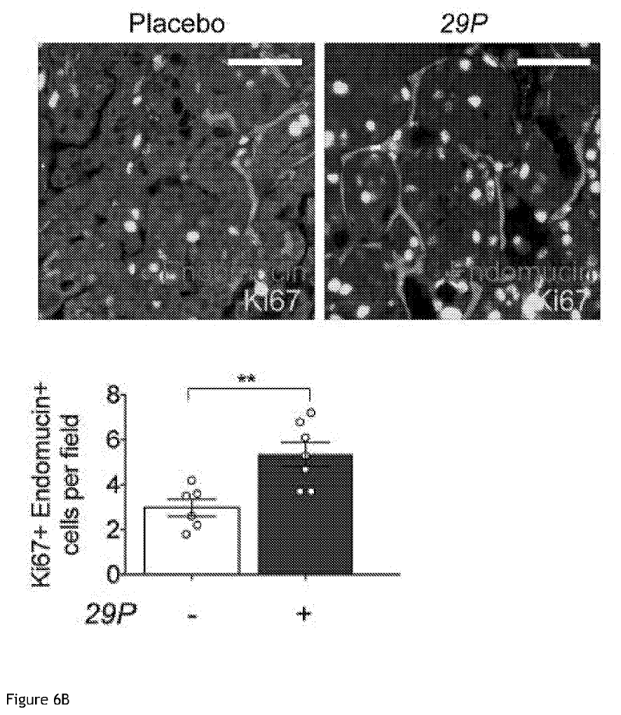

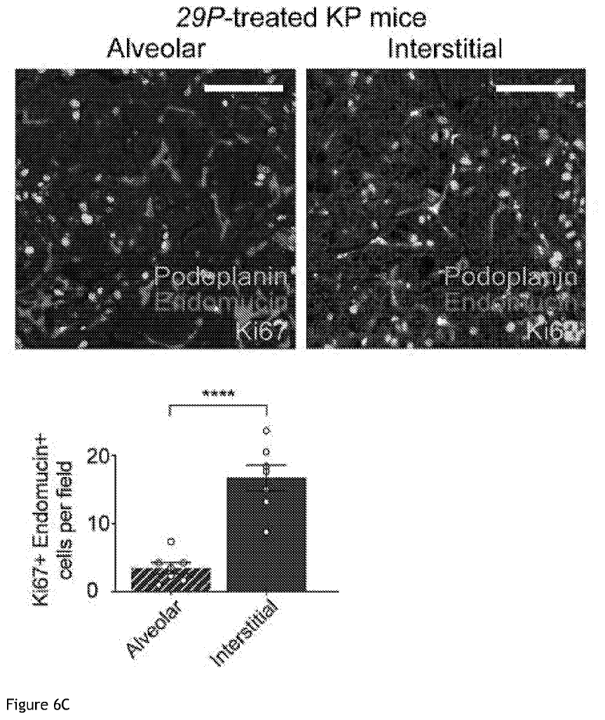

We also investigated the molecular basis of enhanced blood vessel numbers after 29P treatment in tumors with an interstitial growth pattern. Our previous work indicated that treatment with low dose RGD-mimetics enhanced VEGFR2 mediated angiogenesis (26). Here, we show that treatment with 29P in combination with the VEGFR2-inhibitor DC101 resulted in a reduction in the number of tumor foci, tumor burden and blood vessel density. This effect was highly evident in tumors with interstitial growth patterns, but essentially absent in tumors with alveolar growth patterns. This data suggests that tumors with an alveolar growth pattern are resistant to vascular modulation by 29P and that the effects of 29P are VEGFR2 dependent (FIG. 6A).

RNA-seq analysis identified that endothelial cells treated in vitro with 29P showed an upregulation of DNA replication and cell cycle transcripts (see Reactome DNA Replication, Reactome Cell Cycle Mitotic and Reactome Respiratory Electron Transport) and a downregulation of transcripts related to extracellular matrix formation (see Naba Core Matrisome, Naba Basement Membranes, KEGG Focal Adhesion), all processes involved in enhancing angiogenesis. In vivo, picrosirius red analysis identified reduced collagen organization after 29P treatment, reminiscent of the reduced ECM transcript signature in vitro. Additionally, 29P treatment also significantly enhanced the levels of proliferating endothelial cells in vivo (FIG. 6B). After 29P treatment the number of Ki67 positive endothelial cells was significantly higher in tumors with interstitial growth patterns compared with those with alveolar patterns (FIG. 6C). Briefly, 29P induces VEGFR2-dependent angiogenesis preferentially in tumors with interstitial growth pattern and not in the tumors with alveolar growth pattern.

### Example 7—29P Treatment Induces an Anti-Tumor Immune Effect in KP Tumors In Vivo

We also explored what other molecular changes are induced by 29P treatment in vivo. Previous studies have indicated that CXCL9 and INF-γ-related genes are predictors of anti-tumor immune response in NSCLC (27). Moreover, IFN-y-related mRNA profiles can predict clinical response to PD-1 blockade in some cancers such as melanoma (28). CXCL9 is a chemoattractant chemokine that promotes T-cell infiltration into tumors and is induced by IFN-γ (29).

KP tumor bearing lungs, from mice treated with 29P compared with placebo controls, showed an upregulation of RNA-transcript signatures involved in the immune response. 29P treatment enriched pathways including those associated with Cytokine-Cytokine receptor interaction, such as immunogenic cytokines and their receptors, including CXCL13 and its receptor CXCR5, and the chemokines involved in T cell migration: CCL5, CXCL9, CCL10 and their receptor CXCR3 (also receptor for CXCL13). Other cytotoxic pathways such as Interferon-gamma signaling pathways were also significantly upregulated after 29P treatment. Interestingly, IL18 (also known as Interferon inducing factor) is also upregulated after 29P treatment. Corroborating these data, endothelial cells treated with 29P in vitro also showed an upregulation of interferon (IFN-I, IFN-γ) pathway components (Ifr5, Ifit2, Gbp2, Mx1, Rnasel, Stat2, Xaf1, Ifitm3, Oas3, Ifi35, lsg15, Oas2, Psmb8, Irf1, Ifit1, Ifit3, Stat1, lsg20, Usp18). 29P treatment also induced a significant enrichment in reactomes for Leukocyte transmigration and Cell surface receptors at the vascular wall. Together, these data suggest that 29P induces molecular changes that correlate with an enhanced anti-tumor immune response and provide a likely explanation for the enhanced efficacy of anti-PD-1/anti-CTLA-4 treatment we have described in this study.

Importantly, a comparison of upregulated RNA signatures from 29P treated mouse tumor bearing lungs with upregulated transcript signatures from immunoresponsive human cancers (30) identified that CXCL9, CXCL10, CXCL13, CCL8 and CCL5, all major mediators of T cell infiltration, were commonly upregulated suggesting clinical relevance for 29P treatment in enhancing immunotherapy.

### Example 8—Sensitization of the Tumors Induced by 29P is T-cell, IL-18, IFN-γ and CXCL9 Dependent

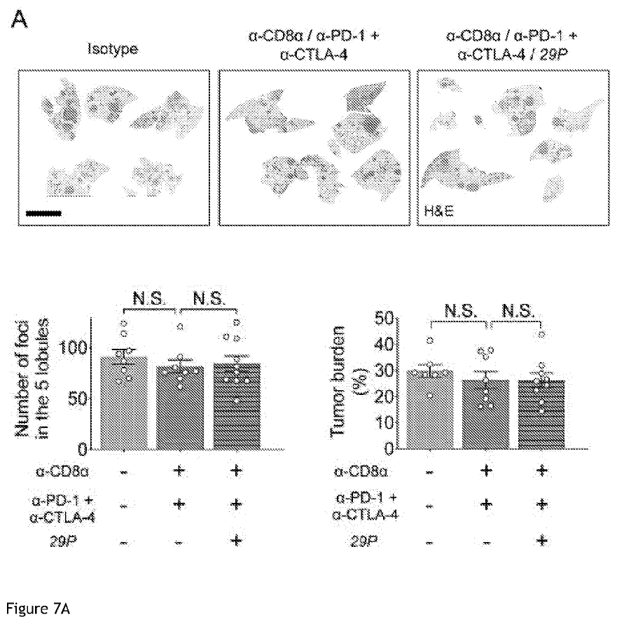

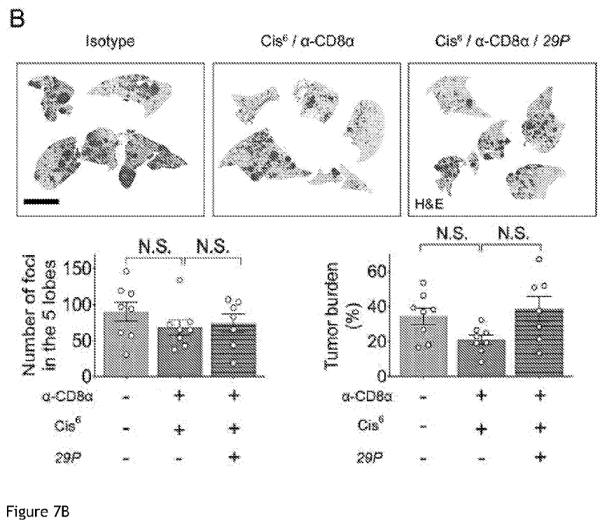

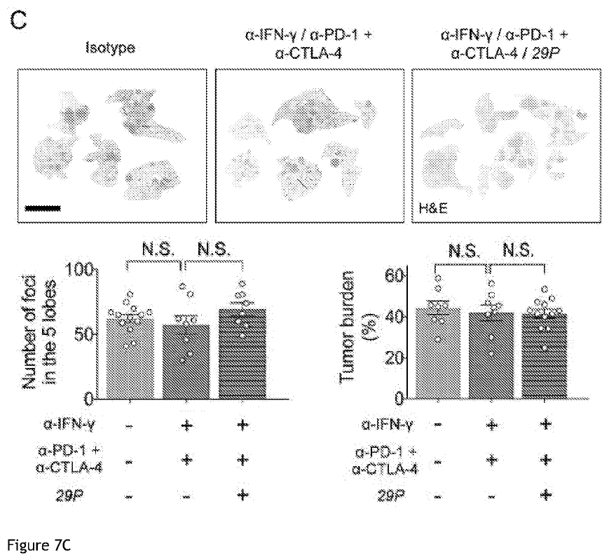

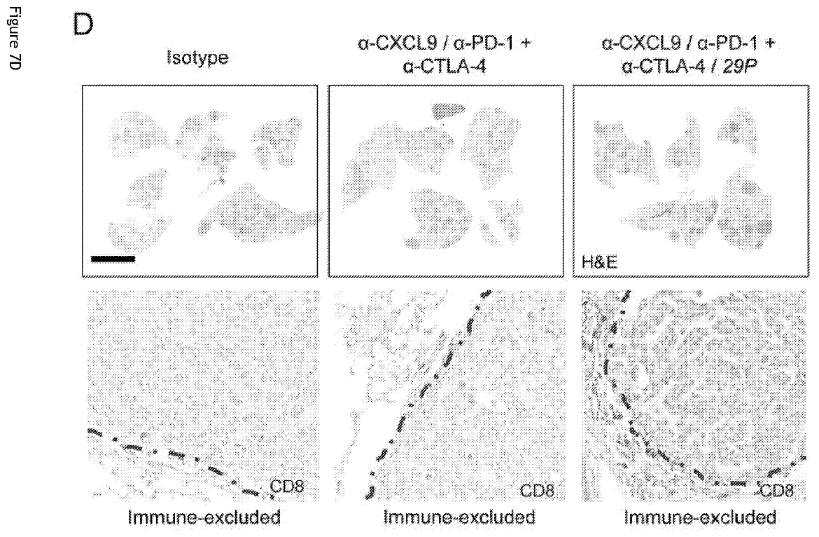

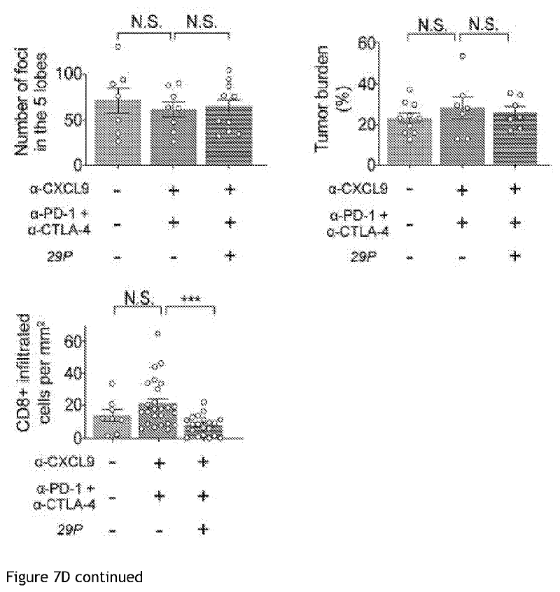

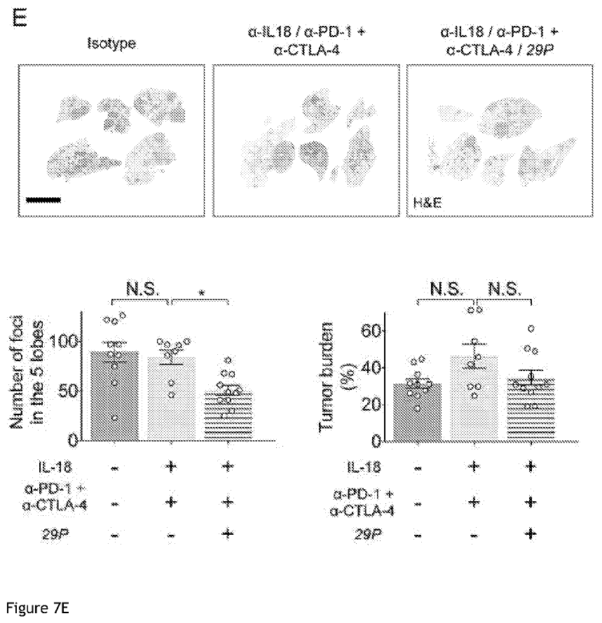

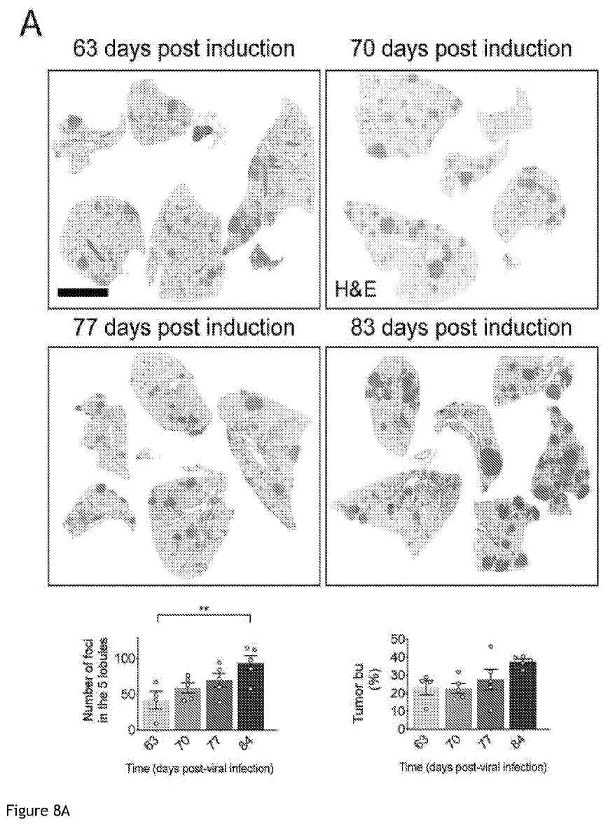

We hypothesized that increased CD8+ cells and IL-18, INFγ and CXCL9 levels hold key functional relevance in the enhanced efficacy of immunotherapy induced by 29P treatment. To investigate this, we performed a series of experiments where we depleted each of these components in combination with our existing therapeutic regimen. CD8+ cell depletion by administration of anti-CD8α antibodies into KP mice treated with the triple combination of anti-PD-1/anti-CTLA-4 antibodies and 29P abolished the treatment effect and mice showed no reduction in the number of foci, tumor area or tumor burden, similar to the placebo treated group (FIG. 7A). Similarly, combination treatment of Cis⁶ and 29P no longer resulted in reduced numbers of KP tumor foci, tumor area or tumor burden when the CD8+ cell population was ablated using anti-CD8x antibody (FIG. 7B). Thus, the effects observed with the combination of cisplatin and 29P in controlling tumor growth are likely due to the promotion of an immunopermissive tumor microenvironment with higher levels of activated CD8+ cells.

Further supporting our RNA sequencing data analysis that IFN-, IL-18, and CXCL9 are crucial mediators of the anti-tumor effects of immunotherapy, when we treated KP tumor-burdened mice with the triple combination of anti-PD-1/anti-CTLA-4 antibodies and 29P and also antibodies which functionally inhibit IFN-γ or CXCL9, animals no longer responded to the immunotherapy and showed no reduction in tumor burden or number of foci. While treatment with the anti-IL-18 antibody in addition to the triple treatment still resulted in a significant reduction in the number of foci, the overall tumor burden remained unchanged compared to placebo (FIG. 7C-E). We demonstrate that the 29P-induced effect on T cell infiltration is impaired when CXCL9 is ablated using neutralizing antibody. Tumors treated with the combination of anti-PD-1/anti-CTLA-4, 29P and anti-CXCL9 show and immune-excluded phenotype, in which the CD8+ remain at the periphery of the different lung tumor lesions, indicating an important function of CXCL9 in the migration of CD8+ to the core of the tumor after 29P treatment (FIG. 7C) Taken together, these data provide strong evidence that treatment with 29P increases levels of IL-18, IFN-γ and CXCL9 which are functionally relevant to the increased efficacy of immunotherapy modulated by 29P treatment.

### Discussion

Clinical trials using immunotherapies in lung cancer, including PDL1 or CTLA-4 antibodies, have shown that this is a very promising first line option with good results in patients with high expression of PD-L1 in the tumor cells (Reck et al). However, this is only true for <30% of all patients diagnosed. For the majority of patients, the heterogeneity of the tumor microenvironment and especially the degree of infiltrating cytotoxic T cells, is a key determinant of the responsiveness to immunotherapy (31).

In this study, we investigated if a vascular promotion strategy would be able to overcome this inherent resistance to immunotherapy and enable us to make NSCLC tumors more susceptible to treatment. To investigate this, we used the gold standard genetically engineered KP mouse model that closely recapitulates both the human disease and the population of patients who do not respond to current therapies. The use of anti-angiogenic therapy in NSCLC patients is still debated. As we demonstrated, KP tumors are largely not angiogenic but instead grow in a vessel co-option mode. These tumors also do not respond to anti-angiogenic therapy, this could explain why many patients have failed to respond to VEGFR2 inhibitors and similar drugs in the clinic.

We modulated the quiescent tumor vasculature of the non-angiogenic vessel co-opting growth patterns (interstitial and alveolar) found in the majority of KP tumors and also in patient samples. This strategy, using the RGD-mimetic hexapeptide 29P, increased blood vessel density in a neo-angiogenic, VEGFR2-dependent mechanism in KP tumors that grow in an interstitial pattern. The fine vessel network promoted in these tumors led to a higher vessel perfusion and also a reduction in hypoxia. It has been previously described that while hypoxic zones inside the tumor mass lack T cells, hypoxia loss reverses T cell exclusion (4). Using 29P, we were able to promote T cell infiltration into KP tumors and thereby sensitize previously immunologically ‘cold’ KP tumors to immune checkpoint blockade.

In this study, we modulated the tumor vasculature in non-angiogenic tumors, and thus targeting quiescent vessels. Vessel co-option is believed to be a mechanism of resistance to anti-angiogenesis, as tumors with co-opting blood vessels are resistant to anti-angiogenic therapy (12). We demonstrated in this study that pro-angiogenic approaches are useful for targeting the quiescent and non-angiogenic tumors. We demonstrate that using 29P we promote neo-angiogenesis in the tumors with interstitial growth pattern, which become sensitized to anti-VEGFR2 treatment, but not in the alveolar growth pattern. The increased blood vessel density after 29P treatment correlates with a decrease in tumor hypoxia and increased CD8+ cell infiltration, thus all correlating with an enhancement of immunotherapy efficacy.

In parallel, our results using RNAseq analysis demonstrated a mechanism based in cytokine production and their receptors, which was also validated functionally, centring on the CXCL9-IL-18-IFN signaling axis. CXCL9, which is involved in T cell migration, and IFN-γ, related to the cytotoxic capacity of the CD8+ cells, or IFN pathways, were all found to be upregulated in endothelial cells after treatment with 29P. These RNA signatures correlate with response to therapy in human cancer patient samples; here, they predicted the response to therapy when we combined chemo- and immunotherapy treatments with 29P.

We have thus provided proof of principle of a new concept to target non-angiogenic tumors, and how this vascular modulation may be of benefit to enhance the efficacy of immunotherapy and chemotherapy. We believe that this approach will functionally affect other immunologically ‘cold’ and non-angiogenic tumors, such as non angiogenic lung metastases or metastases in liver or brain, providing a way to enhance treatment efficacy for a variety of cancers.

### REFERENCES

1. M. Jamal-Hanjani et al., Tracking the Evolution of Non-Small-Cell Lung Cancer. N Engl J Med 376, 2109-2121 (2017).
2. F. R. Hirsch et al., Lung cancer: current therapies and new targeted treatments. Lancet 389, 299-311 (2017).
3. M. Reck, D. Heigener, N. Reinmuth, Immunotherapy for small-cell lung cancer: emerging evidence. Future Oncol 12, 931-943 (2016).
4. P. Jayaprakash et al., Targeted hypoxia reduction restores T cell infiltration and sensitizes prostate cancer to immunotherapy. J Clin Invest 128, 5137-5149 (2018).
5. M. W. Teng, S. F. Ngiow, A. Ribas, M. J. Smyth, Classifying Cancers Based on T-cell Infiltration and PD-L1. Cancer Res 75, 2139-2145 (2015).
6. M. Georganaki, L. van Hooren, A. Dimberg, Vascular Targeting to Increase the Efficiency of Immune Checkpoint Blockade in Cancer. Front Immunol 9, 3081 (2018).
7. R. S. Riley, C. H. June, R. Langer, M. J. Mitchell, Delivery technologies for cancer immunotherapy. Nat Rey Drug Discov, (2019).
8. P. C. Tumeh et al., PD-1 blockade induces responses by inhibiting adaptive immune resistance. Nature 515, 568-571 (2014).
9. R. J. deLeeuw, S. E. Kost, J. A. Kakal, B. H. Nelson, The prognostic value of FoxP3+ tumor-infiltrating lymphocytes in cancer: a critical review of the literature. Clin Cancer Res 18, 3022-3029 (2012).
10. Y. Huang, S. Goel, D. G. Duda, D. Fukumura, R. K. Jain, Vascular normalization as an emerging strategy to enhance cancer immunotherapy. Cancer Res 73, 2943-2948 (2013).
11. E. A. Kuczynski, P. B. Vermeulen, F. Pezzella, R. S. Kerbel, A. R. Reynolds, Vessel co-option in cancer. Nat Rev Clin Oncol 16, 469-493 (2019).
12. V. L. Bridgeman et al., Vessel co-option is common in human lung metastases and mediates resistance to anti-angiogenic therapy in preclinical lung metastasis models. J Pathol 241, 362-374 (2017).
13. E. A. Kuczynski, A. R. Reynolds, Vessel co-option and resistance to anti-angiogenic therapy. Angiogenesis 23, 55-74 (2020).
14. S. Suzuki et al., Interstitial growth as an aggressive growth pattern in primary lung cancer. J Cancer Res Clin Oncol 142, 1591-1598 (2016).
15. D. Fukumura, J. Kloepper, Z. Amoozgar, D. G. Duda, R. K. Jain, Enhancing cancer immunotherapy using antiangiogenics: opportunities and challenges. Nat Rev Clin Oncol 15, 325-340 (2018).
16. A. Alshangiti, G. Chandhoke, P. M. Ellis, Antiangiogenic therapies in non-small-cell lung cancer. Curr Oncol 25, S45-S58 (2018).
17. M. Di Tacchio et al., Tumor Vessel Normalization, Immunostimulatory Reprogramming, and Improved Survival in Glioblastoma with Combined Inhibition of PD-1, Angiopoietin-2, and VEGF. Cancer Immunol Res 7, 1910-1927 (2019).
18. M. Schmittnaegel et al., Dual angiopoietin-2 and VEGFA inhibition elicits antitumor immunity that is enhanced by PD-1 checkpoint blockade. Sci Transl Med 9, (2017).
19. K. A. Khan, R. S. Kerbel, Improving immunotherapy outcomes with anti-angiogenic treatments and vice versa. Nat Rev Clin Oncol 15, 310-324 (2018).
20. P. P. Wong et al., Dual-action combination therapy enhances angiogenesis while reducing tumor growth and spread. Cancer Cell 27, 123-137 (2015).
21. M. Weinmuller et al., Overcoming the Lack of Oral Availability of Cyclic Hexapeptides: Design of a Selective and Orally Available Ligand for the Integrin alphavbeta3. Angew Chem Int Ed Engl 56, 16405-16409 (2017).
22. M. DuPage, A. L. Dooley, T. Jacks, Conditional mouse lung cancer models using adenoviral or lentiviral delivery of Cre recombinase. Nat Protoc 4, 1064-1072 (2009).
23. C. Pfirschke et al., Immunogenic Chemotherapy Sensitizes Tumors to Checkpoint Blockade Therapy. Immunity 44, 343-354 (2016).
T. G. Oliver et al., Chronic cisplatin treatment promotes enhanced damage repair and 24. tumor progression in a mouse model of lung cancer. Genes Dev 24, 837-852 (2010).
25. L. Paz-Ares et al., Pembrolizumab plus Chemotherapy for Squamous Non-Small-Cell Lung Cancer. N Engl J Med 379, 2040-2051 (2018).
26. A. R. Reynolds et al., Stimulation of tumor growth and angiogenesis by low concentrations of RGD-mimetic integrin inhibitors. Nat Med 15, 392-400 (2009).
27. C. A. Hamm, K. Pry, J. Lu, S. Bacus, Immune profiling reveals the diverse nature of the immune response in NSCLC and reveals signaling pathways that may influence the anti-tumor immune response. Exp Mol Pathol 109, 1-15 (2019).
28. M. Ayers et al., IFN-gamma-related mRNA profile predicts clinical response to PD-1 blockade. J Clin Invest 127, 2930-2940 (2017).
29. J. A. Trujillo, R. F. Sweis, R. Bao, J. J. Luke, T Cell-Inflamed versus Non-T Cell-Inflamed Tumors: A Conceptual Framework for Cancer Immunotherapy Drug Development and Combination Therapy Selection. Cancer Immunol Res 6, 990-1000 (2018).
30. S. Mariathasan et al., TGFbeta attenuates tumour response to PD-L1 blockade by contributing to exclusion of T cells. Nature 554, 544-548 (2018).
31. M. Reck et al., Pembrolizumab versus Chemotherapy for PD-L1-Positive Non-Small-Cell Lung Cancer. N Engl J Med 375, 1823-1833 (2016).
32. M. Schmittnaegel, M. De Palma, Reprogramming Tumor Blood Vessels for Enhancing Immunotherapy. Trends Cancer 3, 809-812 (2017).
33. R. K. Jain, Antiangiogenesis strategies revisited: from starving tumors to alleviating hypoxia. Cancer Cell 26, 605-622 (2014).
34. B. Tavora et al., Endothelial-cell FAK targeting sensitizes tumours to DNA-damaging therapy. Nature 514, 112-116 (2014).

1. An αvβ3- and/or αvβ5-integrin targeting agent for use in a method of treating a cancer in a patient, wherein said method further comprises administering at least one immunotherapeutic agent and/or at least one chemotherapeutic agent to the patient.

2. The αvβ3- and/or αvβ5-integrin targeting agent for use according to claim 1, further comprising administering at least one immunotherapeutic agent and at least one chemotherapeutic agent to the patient.

3. The αvβ3- and/or αvβ5-integrin targeting agent for use according to claim 1 or 2, wherein the cancer is a solid cancer.

4. The αvβ3- and/or αvβ5-integrin targeting agent for use according to claim 3, wherein the cancer is lung cancer, optionally non-small cell lung cancer.

5. The avB3- and/or avB5-integrin targeting agent for use according to any one of the preceding claims, where the cancer is non-angiogenic and/or exhibits vessel co-option.

6. The αvβ3- and/or avB5-integrin targeting agent for use according to any one of the preceding claims, wherein the cancer is refractory to immunotherapy.

7. The avB3- and/or avB5-integrin targeting agent for use according to any one of the preceding claims, wherein said method enhances survival of the patient from said cancer.

8. The αvβ3- and/or αvβ5-integrin targeting agent for use according to any one of the preceding claims, wherein administration of the αvβ3- and/or αvβ5-integrin targeting agent increases immune infiltration of the cancer.

9. The αvβ3- and/or avB5-integrin targeting agent for use according to any one of the preceding claims, wherein the αvβ3- and/or αvβ5-integrin targeting agent is a small molecule, a peptide, a peptidomimetic, a protein, or an antibody

10. The αvβ3- and/or αvβ5-integrin targeting agent for use according to claim 9, wherein the αvβ3- and/or αvβ5-integrin targeting agent is a peptide comprising an RGD motif or a peptidomimetic thereof, optionally wherein the peptide or peptidomimetic is a cyclic peptide of about five to about 8 amino acids in length or a peptidomimetic thereof, optionally wherein the peptide or peptidomimetic is a pentapeptide or a hexapeptide or a peptidomimetic thereof.

11. The αvβ3- and/or αvβ5-integrin targeting agent for use according to claim 10, wherein:
(a) the peptide or peptidomimetic is a said peptide comprising at least one N-alkylated amino acid residue, optionally at least one N-methylated amino acid residue, or a peptidomimetic thereof;
(b) wherein the peptide or peptidomimetic is a said peptide comprising at least one D-amino acid or Gly, or a peptidomimetic thereof, preferably wherein said peptide or peptidomimetic comprises at least one βII′ turn and the at least one D-amino acid or Gly is provided at the i+1 position of the βII′ turn; and/or
(c) the peptide or peptidomimetic is a said peptide comprising at least one hydrophobic amino acid residue, optionally selected from Val or Phe, or a peptidomimetic thereof.

12. The αvβ3- and/or αvβ5-integrin targeting agent for use according to claim 10 or 11, wherein the peptide is selected from cilengitide or a derivative or analogue or peptidomimetic thereof, or one of the following peptides or a derivative or analogue or peptidomimetic thereof:

| | a. |
|---|---|
| (SEQ ID NO: 1) | |
| | *rGDA*AA; |
| | b. |
| (SEQ ID NO: 13) | |
| | *GDAA*A; |
| | c. |
| (SEQ ID NO: 2) | |
| | *aRGDA*A; |
| | d. |
| (SEQ ID NO: 3) | |
| | rG*DA*AA; |
| | e. |
| (SEQ ID NO: 4) | |
| | rGDA*A*A; |
| | f. |
| (SEQ ID NO: 5) | |
| | *vRGDA*A; |
| | g. |
| (SEQ ID NO: 6) | |
| | *fRGDA*A; |
| | h. |
| (SEQ ID NO: 7) | |
| | *rGDA*AV; |
| | and |
| | i. |
| (SEQ ID NO: 8) | |
| | *rGDA*AF; |

wherein * represents N-methylation of the following amino acid, R is arginine, G is glycine, D is aspartic acid, r is arginine in the D configuration, A is alanine, a is alanine in the D configuration, V is valine, v is valine in the D configuration, F is phenylalanine, and f is phenylalanine in the D configuration, and preferably the peptide is a cyclic peptide of any one of SEQ ID NOs 24-31 and 36, or a derivative or analogue or peptidomimetic thereof.

13. The αvβ3- and/or αvβ5-integrin targeting agent for use according to claim 12, wherein the peptide is *vRGDA*A (SEQ ID NO: 5), a cyclic peptide of SEQ ID NO: 28, or a derivative or analogue or peptidomimetic of either thereof.

14. The αvβ3- and/or αvβ5-integrin targeting agent for use according to any one of claims 9 to 13, wherein the peptide or peptidomimetic is in the form of a prodrug, or is provided as a conjugate or fusion protein.

15. The αvβ3- and/or αvβ5-integrin targeting agent for use according to claim 14, wherein the prodrug comprises at least one moiety that reduces net charge of the peptide or peptidomimetic, and/or comprises at least one of the following moieties or groups of moieties:
(i) a —CO₂R moiety, wherein R is alkyl;
(ii) a hexyloxycarbonyl (Hoc) moiety, optionally linked to an arginine residue;
(iii) a methyl ester moiety (OMe)or other alkyl ester moiety, such as a methyl or alkyl ester of Asp;
(iv) hexyloxycarbonyl (Hoc) and ester (OMe) moieties; and/or
(v) two hexyloxycarbonyl (Hoc) moieties;
optionally wherein the prodrug has the formula: c(*vR(Hoc)₂GD(OMe)A*A)(SEQ ID NO: 9), c(*aR(Hoc)₂GD(OMe)A*A)(SEQ ID NO: 10), c(*r(Hoc)₂GD(OMe)A*AA)(SEQ ID NO: 11) or c(r(Hoc)₂GD(OMe)A*A*A)(SEQ ID NO: 12).

16. The αvβ3- and/or αvβ5-integrin targeting agent for use according to any one of claims 9 to 15, wherein the small molecule, peptide or peptidomimetic is administered orally.

17. The αvβ3- and/or αvβ5-integrin targeting agent for use according to any one of claims 9 to 16, wherein the peptide or peptidomimetic is administered as a pharmaceutical composition comprising said peptide or peptidomimetic, a pharmaceutically acceptable carrier, excipient or diluent.

18. The αvβ3- and/or avB5-integrin targeting agent for use according to any one of the preceding claims, wherein the at least one immunotherapeutic agent induces a CD8+ T cell anti-cancer response.

19. The αvβ3- and/or avB5-integrin targeting agent for use according to any one of the preceding claims, wherein the at least one immunotherapeutic agent comprises one or more checkpoint inhibitors, optionally wherein a said checkpoint inhibitor targets the interaction of PD-1, CTLA-4 or PD-L1 with a ligand.

20. The αvβ3- and/or avB5-integrin targeting agent for use according to claim 19, wherein a said checkpoint inhibitor is an antibody or CAR-T cell, optionally an antibody or CAR-T cell binding PD1, PD-L1 or CTLA4.

21. The αvβ3- and/or αvβ5-integrin targeting agent for use according to claim 2 or any one of claims 3-20 as dependent on claim 2, wherein the at least one chemotherapeutic agent comprises at least one alkylating agent, optionally cisplatin, carboplatin or a derivative of any thereof.

22. At least one immunotherapeutic agent for use in a method of treating a cancer in a patient, wherein said method further comprises administering an αvβ3- and/or αvβ5-integrin targeting agent to the patient, optionally wherein the at least one immunotherapeutic agent is as defined in any one of claims 18-20 and/or the αvβ3- and/or αvβ5-integrin targeting agent is as defined in any one of claims 8-15.

23. A method of treating a cancer in a patient, comprising administering an αvβ3- and/or αvβ5-integrin targeting agent and at least one immunotherapeutic agent, optionally wherein the at least one immunotherapeutic agent is as defined in any one of claims 18-20 and/or the αvβ3- and/or αvβ5-integrin targeting agent is as defined in any one of claims 8-15.

24. Use of an αvβ3- and/or αvβ5-integrin targeting agent in the manufacture of a medicament for treatment of cancer, wherein the treatment further comprises administration of at least one immunotherapeutic agent, optionally wherein the at least one immunotherapeutic agent is as defined in any one of claims 18-20 and/or the αvβ3- and/or avB5-integrin targeting agent is as defined in any one of claims 8-15.

25. A combination of an αvβ3- and/or αvβ5-integrin targeting agent and at least one immunotherapeutic agent and/or at least one chemotherapeutic agent, optionally wherein, optionally wherein the αvβ3- and/or αvβ5-integrin targeting agent is as defined in any one of claims 8-15, the at least one immunotherapeutic agent is as defined in any one of claims 18-20, and/or the at least one chemotherapeutic agent is as defined in claim 21.

26. An αvβ3- and/or αvβ5-integrin targeting agent for use in a method of treating a cancer in a patient, wherein said method further comprises administering at least one chemotherapeutic agent, optionally wherein the αvβ3- and/or αvβ5-integrin targeting agent is as defined in any one of claims 8-15, the cancer is as defined in claim 4 or 5 and/or the at least one chemotherapeutic agent is as defined in claim 21.


SEQUENCE LISTING (46 sequences)

SEQ ID NO: 1 - protein, 6 aa, Artificial Sequence
  Synthetic peptide
  MISC_FEATURE (1)..(1): D-amino acid
  MISC_FEATURE (1)..(1): N-methylated
  MISC_FEATURE (5)..(5): N-methylated
RGDAAA

SEQ ID NO: 2 - protein, 6 aa, Artificial Sequence
  Synthetic peptide
  MISC_FEATURE (1)..(1): D-amino acid
  MISC_FEATURE (1)..(1): N methylated
  MISC_FEATURE (6)..(6): N methylated
ARGDAA

SEQ ID NO: 3 - protein, 6 aa, Artificial Sequence
  Synthetic peptide
  MISC_FEATURE (1)..(1): D-amino acid
  MISC_FEATURE (3)..(3): N methylated
  MISC_FEATURE (5)..(5): N methylated
RGDAAA

SEQ ID NO: 4 - protein, 6 aa, Artificial Sequence
  Synthetic peptide
  MISC_FEATURE (1)..(1): D-amino acid
  MISC_FEATURE (5)..(5): N methylated
  MISC_FEATURE (6)..(6): N methylated
RGDAAA

SEQ ID NO: 5 - protein, 6 aa, Artificial Sequence
  Synthetic peptide
  MISC_FEATURE (1)..(1): D-amino acid
  MISC_FEATURE (1)..(1): N methylated
  MISC_FEATURE (6)..(6): N methylated
VRGDAA

SEQ ID NO: 6 - protein, 6 aa, Artificial Sequence
  Synthetic peptide
  MISC_FEATURE (1)..(1): D-amino acid
  MISC_FEATURE (1)..(1): N methylated
  MISC_FEATURE (6)..(6): N methylated
FRGDAA

SEQ ID NO: 7 - protein, 6 aa, Artificial Sequence
  Synthetic peptide
  MISC_FEATURE (1)..(1): D-amino acid
  MISC_FEATURE (1)..(1): N methylated
  MISC_FEATURE (5)..(5): N methylated
RGDAAV

SEQ ID NO: 8 - protein, 6 aa, Artificial Sequence
  Synthetic peptide
  MISC_FEATURE (1)..(1): D-amino acid
  MISC_FEATURE (1)..(1): N methylated
  MISC_FEATURE (5)..(5): N methylated
RGDAAF

SEQ ID NO: 9 - protein, 6 aa, Artificial Sequence
  Cyclic peptide
  MISC_FEATURE (1)..(1): D-amino acid
  MISC_FEATURE (1)..(1): N methylated
  MISC_FEATURE (2)..(2): Two hexyloxycarbonyl (Hoc) moieties
  MISC_FEATURE (4)..(4): Methyl ester (OMe) moiety
  MISC_FEATURE (6)..(6): N methylated
VRGDAA

SEQ ID NO: 10 - protein, 6 aa, Artificial Sequence
  Cyclic peptide
  MISC_FEATURE (1)..(1): D-amino acid
  MISC_FEATURE (1)..(1): N methylated
  MISC_FEATURE (2)..(2): Two hexyloxycarbonyl (Hoc) moieties
  MISC_FEATURE (4)..(4): Methyl ester (OMe) moiety
  MISC_FEATURE (6)..(6): N methylated
ARGDAA

SEQ ID NO: 11 - protein, 6 aa, Artificial Sequence
  Cyclic peptide
  MISC_FEATURE (1)..(1): D-amino acid
  MISC_FEATURE (1)..(1): N methylated
  MISC_FEATURE (1)..(1): Two hexyloxycarbonyl (Hoc) moieties
  MISC_FEATURE (3)..(3): Methyl ester (OMe) moiety
  MISC_FEATURE (5)..(5): N methylated
RGDAAA

SEQ ID NO: 12 - protein, 6 aa, Artificial Sequence
  Cyclic peptide
  MISC_FEATURE (1)..(1): D-amino acid
  MISC_FEATURE (1)..(1): Two hexyloxycarbonyl (Hoc) moieties
  MISC_FEATURE (3)..(3): Methyl ester (OMe) moiety
  MISC_FEATURE (5)..(5): N methylated
  MISC_FEATURE (6)..(6): N methylated
RGDAAA

SEQ ID NO: 13 - protein, 6 aa, Artificial Sequence
  Synthetic peptide
  MISC_FEATURE (1)..(1): D-amino acid
  MISC_FEATURE (1)..(1): N methylated
  MISC_FEATURE (6)..(6): N methylated
RGDAAA

SEQ ID NO: 14 - protein, 6 aa, Artificial Sequence
  Synthetic peptide
  MISC_FEATURE (1)..(1): D-amino acid
  MISC_FEATURE (1)..(1): N methylated
  MISC_FEATURE (2)..(2): Two hexyloxycarbonyl (Hoc) moieties
  MISC_FEATURE (4)..(4): Methyl ester (OMe) moiety
  MISC_FEATURE (6)..(6): N methylated
FRGDAA

SEQ ID NO: 15 - protein, 9 aa, Artificial Sequence
  Synthetic peptide
  MISC_FEATURE (5)..(5): Two hexyloxycarbonyl (Hoc) moieties
LPPFRGDLA

SEQ ID NO: 16 - protein, 8 aa, Artificial Sequence
  Synthetic peptide
  MISC_FEATURE (6)..(6): Two hexyloxycarbonyl (Hoc) moieties
LPPGLRGD

SEQ ID NO: 17 - protein, 9 aa, Artificial Sequence
  Synthetic peptide
LPPFRGDLA

SEQ ID NO: 18 - protein, 8 aa, Artificial Sequence
  Synthetic peptide
LPPGLRGD

SEQ ID NO: 19 - protein, 6 aa, Artificial Sequence
  Synthetic peptide
  MISC_FEATURE (1)..(1): D-amino acid
AAAAAA

SEQ ID NO: 20 - protein, 5 aa, Artificial Sequence
  Synthetic peptide
  MISC_FEATURE (1)..(1): D-amino acid
  MISC_FEATURE (2)..(2): N methylated
FVRGD

SEQ ID NO: 21 - protein, 6 aa, Artificial Sequence
  Synthetic peptide
  MISC_FEATURE (1)..(1): D-amino acid
  MISC_FEATURE (1)..(1): N methylated
  MISC_FEATURE (2)..(2): Hexyloxycarbonyl (Hoc) moiety
  MISC_FEATURE (4)..(4): Methyl ester (OMe) moiety
  MISC_FEATURE (6)..(6): N methylated
VRGDAA

SEQ ID NO: 22 - protein, 6 aa, Artificial sequence
  Synthetic peptide
  MISC_FEATURE (1)..(1): D-amino acid
  MISC_FEATURE (2)..(2): N methylayed
  MISC_FEATURE (3)..(3): Two hexyloxycarbonyl (Hoc) moieties
AAAAAA

SEQ ID NO: 23 - protein, 5 aa, Artificial sequence
  Synthetic peptide
  MISC_FEATURE (1)..(1): D-amino acid
  MISC_FEATURE (2)..(2): N methylated
  MISC_FEATURE (3)..(3): Two hexyloxycarbonyl (Hoc) moieties
FVRGD

SEQ ID NO: 24 - protein, 6 aa, Artificial Sequence
  Cyclic peptide
  MISC_FEATURE (1)..(1): D-amino acid
  MISC_FEATURE (1)..(1): N-methylated
  MISC_FEATURE (5)..(5): N-methylated
RGDAAA

SEQ ID NO: 25 - protein, 6 aa, Artificial Sequence
  Cyclic peptide
  MISC_FEATURE (1)..(1): D-amino acid
  MISC_FEATURE (1)..(1): N methylated
  MISC_FEATURE (6)..(6): N methylated
ARGDAA

SEQ ID NO: 26 - protein, 6 aa, Artificial Sequence
  Cyclic peptide
  MISC_FEATURE (1)..(1): D-amino acid
  MISC_FEATURE (3)..(3): N methylated
  MISC_FEATURE (5)..(5): N methylated
RGDAAA

SEQ ID NO: 27 - protein, 6 aa, Artificial Sequence
  Cyclic peptide
  MISC_FEATURE (1)..(1): D-amino acid
  MISC_FEATURE (5)..(5): N methylated
  MISC_FEATURE (6)..(6): N methylated
RGDAAA

SEQ ID NO: 28 - protein, 6 aa, Artificial Sequence
  Cyclic peptide
  MISC_FEATURE (1)..(1): D-amino acid
  MISC_FEATURE (1)..(1): N methylated
  MISC_FEATURE (6)..(6): N methylated
VRGDAA

SEQ ID NO: 29 - protein, 6 aa, Artificial Sequence
  Cyclic peptide
  MISC_FEATURE (1)..(1): D-amino acid
  MISC_FEATURE (1)..(1): N methylated
  MISC_FEATURE (6)..(6): N methylated
FRGDAA

SEQ ID NO: 30 - protein, 6 aa, Artificial Sequence
  Cyclic peptide
  MISC_FEATURE (1)..(1): D-amino acid
  MISC_FEATURE (1)..(1): N methylated
  MISC_FEATURE (5)..(5): N methylated
RGDAAV

SEQ ID NO: 31 - protein, 6 aa, Artificial Sequence
  Cyclic peptide
  MISC_FEATURE (1)..(1): D-amino acid
  MISC_FEATURE (1)..(1): N methylated
  MISC_FEATURE (5)..(5): N methylated
RGDAAF

SEQ ID NO: 32 - protein, 6 aa, Artificial Sequence
  Synthetic peptide
  MISC_FEATURE (1)..(1): D-amino acid
  MISC_FEATURE (1)..(1): N methylated
  MISC_FEATURE (2)..(2): Two hexyloxycarbonyl (Hoc) moieties
  MISC_FEATURE (4)..(4): Methyl ester (OMe) moiety
  MISC_FEATURE (6)..(6): N methylated
VRGDAA

SEQ ID NO: 33 - protein, 6 aa, Artificial Sequence
  Synthetic peptide
  MISC_FEATURE (1)..(1): D-amino acid
  MISC_FEATURE (1)..(1): N methylated
  MISC_FEATURE (2)..(2): Two hexyloxycarbonyl (Hoc) moieties
  MISC_FEATURE (4)..(4): Methyl ester (OMe) moiety
  MISC_FEATURE (6)..(6): N methylated
ARGDAA

SEQ ID NO: 34 - protein, 6 aa, Artificial Sequence
  Synthetic peptide
  MISC_FEATURE (1)..(1): D-amino acid
  MISC_FEATURE (1)..(1): N methylated
  MISC_FEATURE (1)..(1): Two hexyloxycarbonyl (Hoc) moieties
  MISC_FEATURE (3)..(3): Methyl ester (OMe) moiety
  MISC_FEATURE (5)..(5): N methylated
RGDAAA

SEQ ID NO: 35 - protein, 6 aa, Artificial Sequence
  Synthetic peptide
  MISC_FEATURE (1)..(1): D-amino acid
  MISC_FEATURE (1)..(1): Two hexyloxycarbonyl (Hoc) moieties
  MISC_FEATURE (3)..(3): Methyl ester (OMe) moiety
  MISC_FEATURE (5)..(5): N methylated
  MISC_FEATURE (6)..(6): N methylated
RGDAAA

SEQ ID NO: 36 - protein, 6 aa, Artificial Sequence
  Cyclic peptide
  MISC_FEATURE (1)..(1): D-amino acid
  MISC_FEATURE (1)..(1): N methylated
  MISC_FEATURE (6)..(6): N methylated
RGDAAA

SEQ ID NO: 37 - protein, 6 aa, Artificial Sequence
  Cyclic peptide
  MISC_FEATURE (1)..(1): D-amino acid
  MISC_FEATURE (1)..(1): N methylated
  MISC_FEATURE (2)..(2): Two hexyloxycarbonyl (Hoc) moieties
  MISC_FEATURE (4)..(4): Methyl ester (OMe) moiety
  MISC_FEATURE (6)..(6): N methylated
FRGDAA

SEQ ID NO: 38 - protein, 9 aa, Artificial Sequence
  Cyclic peptide
  MISC_FEATURE (5)..(5): Two hexyloxycarbonyl (Hoc) moieties
LPPFRGDLA

SEQ ID NO: 39 - protein, 8 aa, Artificial Sequence
  Cyclic peptide
  MISC_FEATURE (6)..(6): Two hexyloxycarbonyl (Hoc) moieties
LPPGLRGD

SEQ ID NO: 40 - protein, 9 aa, Artificial Sequence
  Cyclic peptide
LPPFRGDLA

SEQ ID NO: 41 - protein, 8 aa, Artificial Sequence
  Cyclic peptide
LPPGLRGD

SEQ ID NO: 42 - protein, 6 aa, Artificial Sequence
  Cyclic peptide
  MISC_FEATURE (1)..(1): D-amino acid
AAAAAA

SEQ ID NO: 43 - protein, 5 aa, Artificial Sequence
  Cyclic peptide
  MISC_FEATURE (1)..(1): D-amino acid
  MISC_FEATURE (2)..(2): N methylated
FVRGD

SEQ ID NO: 44 - protein, 6 aa, Artificial Sequence
  Cyclic peptide
  MISC_FEATURE (1)..(1): D-amino acid
  MISC_FEATURE (1)..(1): N methylated
  MISC_FEATURE (2)..(2): Hexyloxycarbonyl (Hoc) moiety
  MISC_FEATURE (4)..(4): Methyl ester (OMe) moiety
  MISC_FEATURE (6)..(6): N methylated
VRGDAA

SEQ ID NO: 45 - protein, 6 aa, Artificial sequence
  Cyclic peptide
  MISC_FEATURE (1)..(1): D-amino acid
  MISC_FEATURE (2)..(2): N methylayed
  MISC_FEATURE (3)..(3): Two hexyloxycarbonyl (Hoc) moieties
AAAAAA

SEQ ID NO: 46 - protein, 5 aa, Artificial sequence
  Cyclic peptide
  MISC_FEATURE (1)..(1): D-amino acid
  MISC_FEATURE (2)..(2): N methylated
  MISC_FEATURE (3)..(3): Two hexyloxycarbonyl (Hoc) moieties
FVRGD